(19) United States
(12) Patent Application Publication
(10) Pub. No.: US 2015/0239880 A1
(43) Pub. Date: Aug. 27, 2015

(54) LIGHT EMITTING MATERIAL, COMPOUND, AND ORGANIC LIGHT EMITTING DEVICE USING THE SAME

(71) Applicant: KYUSHU UNIVERSITY NATIONAL UNIVERSITY CORP., Fukuoka-shi, Fukuoka (JP)

(72) Inventors: Chihaya Adachi, Fukuoka-shi (JP); Katsuyuki Shizu, Fukuoka-shi (JP); Yuta Sagara, Fukuoka-shi (JP)

(21) Appl. No.: 14/424,138

(22) PCT Filed: Aug. 23, 2013
(86) PCT No.: PCT/JP2013/072499

§ 371 Date: Feb. 26, 2015

(30) Foreign Application Priority Data
Aug. 30, 2012 (JP) 2012-190362

(51) Int. Cl. C07D 417/10; C07D 413/10; C09K 11/06

(52) U.S. Cl. CPC C07D 417/10; C09K 11/06; C07D 413/10; C09K 2211/1018

(57) ABSTRACT

To provide a material useful as a light emitting material of an organic light emitting device. A light emitting material comprising a compound represented by the general formula (1) wherein X represents O, S, N—R¹¹, C═O, C(R¹²)(R¹³), or Si(R¹⁴)(R¹⁵); Y represents O, S, or N—R¹⁶; Ar¹ represents a substituted or unsubstituted arylene group, and Ar² represents an aromatic ring or a heteroaromatic ring; and R¹ to R⁸ and R¹¹ to R¹⁶ each independently represent a hydrogen atom or a substituent.

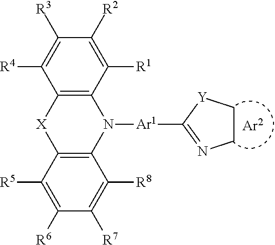

### TECHNICAL FIELD

The present invention relates to a compound that is useful as a light emitting material, and an organic light emitting device using the same.

### BACKGROUND ART

An organic light emitting device, such as an organic electroluminescent device (organic EL device), has been actively studied for enhancing the light emission efficiency thereof. In particular, various studies for enhancing the light emitting efficiency have been made by newly developing and combining an electron transporting material, a hole transporting material, a light emitting material and the like constituting an organic electroluminescent device.

There are studies relating to an organic electroluminescent device utilizing a compound containing a phenazine structure. For example, Patent Reference 1 describes the use of a compound containing a phenazine structure represented by the following general formula as a host material of an organic electroluminescent device and the like. In the general formula, R₁ to R₈ each represent a hydrogen atom, an alkyl group, an aryl group or the like, and R₉ and R₁₀ each represent a hydrogen atom, an alkyl group, an aryl group, a heterocyclic group or an alkenyl group. However, there is no description regarding a benzoxazolyl phenyl group, a benzothiazolyl phenyl group, and an indazolyl phenyl group for R₉ and R₁₀.

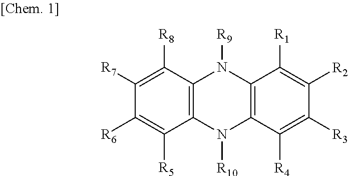

In addition, a compound containing the following structure has been known as a compound that contains a benzoxazolyl phenyl group, a benzothiazolyl phenyl group, or an indazolyl phenyl group. However, such a compound contains a diphenylamino group at a donor site, and there is no description regarding a phenazine structure, a phenoxazine structure, and a phenothiazine structure.

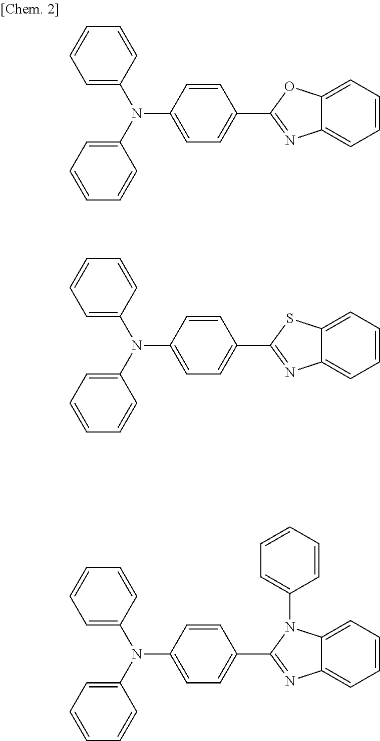 [SMILES: c1ccc(N(c2ccccc2)c2ccc(-c3nc4ccccc4o3)cc2)cc1 ; c1ccc(N(c2ccccc2)c2ccc(-c3nc4ccccc4s3)cc2)cc1 ; c1ccc(N(c2ccccc2)c2ccc(-c3nc4ccccc4n3-c3ccccc3)cc2)cc1]

Patent Reference 2 describes that a compound represented by the following general formula is useful as a host material.

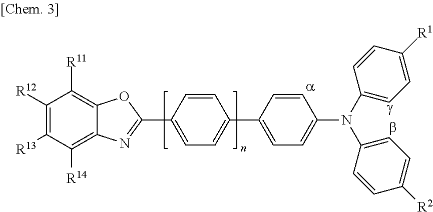

In the above general formula, R¹ and R² each represent a hydrogen atom, an alkyl group having from 1 to 4 carbon atoms, or a substituted or unsubstituted aryl group having from 6 to 13 carbon atoms, R¹¹ to R¹⁴ each represent a hydrogen atom, halogen, an alkyl group having from 1 to 4 carbon atoms, or an unsubstituted aryl group having from 6 to 10 carbon atoms, any two among α, β, and γ are combined to form a bond, a carbazole skeleton is formed, and n is from 0 to 3. Patent Reference 2 also describes a light emitting device that use, as a host material, a compound containing the following structure as a specific compound included in the aforementioned general formula, and there is also a description that no light emission was observed from the compound.

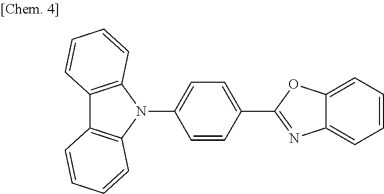

### CITATION LIST

### Patent References

[Patent Reference 1] U.S. Pat. No. 6,869,699

### [Patent Reference 2] JP-A-2010-83862

### SUMMARY OF INVENTION

### Technical Problem

As described above, in relation to the compound containing a phenazine structure or the compound containing a benzoxazolyl phenyl group, a benzothiazolyl phenyl group, or an indazolyl phenyl group, there are known compounds. However, a compound containing a group with a phenazine structure, a phenoxazine structure, or a phenothiazine structure along with a benzoxazolyl phenyl group, a benzothiazolyl phenyl group, or an indazolyl phenyl group has almost not been specifically studied. Such a compound has not been reported even for a synthesis example thereof. Accordingly, it is extremely difficult to predict accurately properties that are exhibited by the compound having a combination of these groups. In particular, for the usefulness thereof as a light emitting material, it is difficult to find any literature capable of becoming the basis of prediction of the usefulness as a light emitting material.

The present inventors have performed investigations with the aim of synthesizing a compound containing in the molecule thereof a phenazine structure, a phenoxazine structure, a phenothiazine structure or the like along with a benzoxazolyl phenyl group, a benzothiazolyl phenyl group, an indazolyl phenyl group, or the like and evaluating the compound for usefulness as a light emitting material in consideration of the above problems in the related art. The inventors have further performed earnest investigations with the aim of evolving a general formula of a compound that is useful as a light emitting material, and generalizing a structure of an organic light emitting device having a high light emission efficiency.

### Solution to Problem

As a result of earnest investigations for achieving the objects, the inventors have succeeded at synthesis of a targeted compound, and have first revealed that the compound is useful as a light emitting material. It has been also found that a compound that is useful as a delayed fluorescent emitter is included in the compound, and the inventors have revealed that an organic light emitting device having a high light emission efficiency may be provided inexpensively. Based on the knowledge, the inventors have consequently provide the invention below as a measure for solving the problems.

[1] A light emitting material comprising: a compound represented by the following general formula (1):

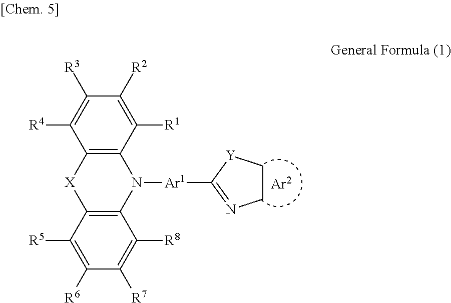

wherein in the general formula (1), X represents O, S, N—R¹¹, C═O, C(R¹²)(R¹³), or Si(R¹⁴)(R¹⁵), and Y represents O, S, or N—R¹⁶. Ar¹ represents a substituted or unsubstituted arylene group, and Ar² represents an aromatic ring or a heteroaromatic ring. R¹ to R⁸ and R¹¹ to R¹⁶ each independently represent a hydrogen atom or a substituent, provided that R¹ and R², R² and R³, R³ and R⁴, R⁵ and R⁶, R⁶ and R⁷, R⁷ and R⁸ each may be bonded to each other to form a cyclic structure.

[2] The light emitting material according to the item [1], wherein the compound represented by the general formula (1) is a compound represented by the following general formula (2):

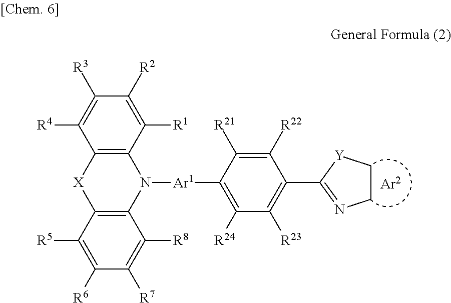

wherein in the general formula (2), X represents O, S, N—R¹¹, C═O, C(R¹²)(R¹³), or Si(R¹⁴)(R¹⁵), and Y represents O, S, or N—R¹⁶. Ar² represents an aromatic ring or a heteroaromatic ring. R¹ to R⁸, R¹¹ to R¹⁶, and R²¹ to R²⁴ each independently represent a hydrogen atom or a substituent, provided that R¹ and R², R² and R³, R³ and R⁴, R⁵ and R⁶, R⁶ and R⁷, R⁷ and R⁸, R²¹ and R²², and R²³ and R²⁴ each may be bonded to each other to form a cyclic structure.

[3] The light emitting material according to the item [1], wherein the compound represented by the general formula (1) is a compound represented by the following general formula (3):

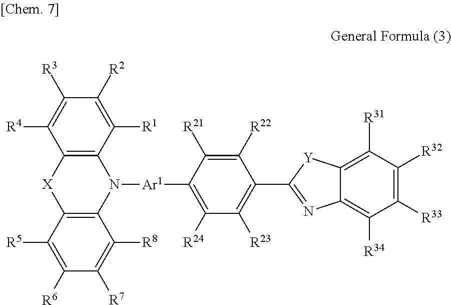

wherein in the general formula (3), X represents O, S, N—R¹¹, C═O, C(R¹²)(R¹³), or Si(R¹⁴)(R¹⁵), and Y represents O, S, or N—R¹⁶. R¹ to R⁸, R¹¹ to R¹⁶, R²¹ to R²⁴, and R³¹ to R³⁴ each independently represent a hydrogen atom or a substituent, provided that R¹ and R², R² and R³, R³ and R⁴, R⁵ and R⁶, R⁶ and R⁷, R⁷ and R⁸, R²¹ and R²², R²³ and R²⁴, R³¹ and R³², R³² and R³³, and R³³ and R³⁴ each may be bonded to each other to form a cyclic structure.

[4] The light emitting material according to any one of the items [1] to [3], wherein X is O or S.

[5] The light emitting material according to any one of the items [1] to [4], wherein Y is 0, 5, or N—R¹⁶, and R¹⁶ represents a substituted or an unsubstituted aryl group.

[6] The light emitting material according to any one of the items [1] to [5], wherein R¹ to R⁸ each independently represent a hydrogen atom, a fluorine atom, a chlorine atom, a cyano group, a substituted or unsubstituted alkyl group having from 1 to 10 carbon atoms, a substituted or unsubstituted alkoxy group having from 1 to 10 carbon atoms, a substituted or unsubstituted dialkylamino group having from 1 to 10 carbon atoms, a substituted or unsubstituted diarylamino group having from 12 to 40 carbon atoms, a substituted or unsubstituted aryl group having from 6 to 15 carbon atoms, or a substituted or unsubstituted heteroaryl group having from 3 to 12 carbon atoms.

[7] A delayed fluorescent emitter comprising the light emitting material according to any one of the items [1] to [6].

[8] A compound represented by the following general formula (1′).

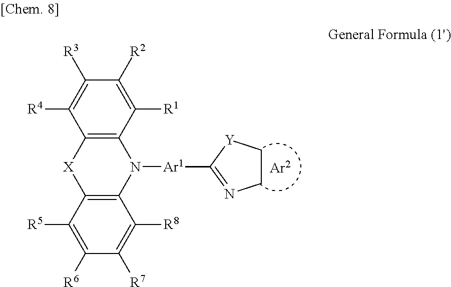

wherein in the general formula (1′), X represents O, S, N—R¹¹, C═O, C(R¹²)(R¹³), or Si(R¹⁴)(R¹⁵), and Y represents O, S, or N—R¹⁶. Ar¹ represents a substituted or unsubstituted arylene group, and Ar² represents an aromatic ring or a heteroaromatic ring. R¹ to R⁸ and R¹¹ to R¹⁶ each independently represent a hydrogen atom or a substituent, provided that R¹⁶ is not a phenyl group when X is O, and R¹ and R², R² and R³, R³ and R⁴, R⁵ and R⁶, R⁶ and R⁷, R⁷ and R⁸ each may be bonded to each other to form a cyclic structure.

[9] An organic light emitting device containing a substrate having thereon a light emitting layer that contains the light emitting material according to any one of the items [1] to [6].

[10] The organic light emitting device according to the item [9], wherein the device emits delayed fluorescent light.

[11] The organic light emitting device according to the item [9] or [10], wherein the device is an organic electroluminescent device.

### Advantageous Effects of Invention

The compound represented by the general formula (1) is useful as a light emitting material. The compound represented by the general formula (1) includes a compound that emits delayed fluorescent light. An organic light emitting device using the compound represented by the general formula (1) as a light emitting material may achieve a high light emission efficiency.

### BRIEF DESCRIPTION OF DRAWINGS

FIG. 1 is a schematic cross sectional view showing an example of a layer structure of an organic electroluminescent device.

FIG. 2 is a ¹H NMR spectrum of a compound 1 synthesized in Synthesis Example 1.

FIG. 3 is a ¹H NMR spectrum of a compound 2 synthesized in Synthesis Example 2.

FIG. 4 is a light emission spectrum of a toluene solution of a compound 1 of Example 1.

FIG. 5 is a transient decay curve of a toluene solution of the compound 1 of Example 1.

FIG. 6 a light emission spectrum of a toluene solution of a compound 2 of Example 1.

FIG. 7 is a transient decay curve of the toluene solution of the compound 2 of Example 1.

FIG. 8 is a light emission spectrum of a toluene solution of a compound 3 of Example 1.

FIG. 9 is a transient decay curve of the toluene solution of the compound 3 of Example 1.

FIG. 10 is a transient decay curve of a toluene solution of a comparative compound in Comparative Example 1.

FIG. 11 is a light emission spectrum of a thin film organic photoluminescent device using a compound 1 of Example 2.

FIG. 12 is a transient decay curve of a thin film organic photoluminescent device using the compound 1 of Example 2.

FIG. 13 is a light emission spectrum of a thin film organic photoluminescent device using a compound 2 of Example 2.

FIG. 14 is a transient decay curve of the thin film organic photoluminescent device using the compound 2 of Example 2.

FIG. 15 is a light emission spectrum of a thin film organic photoluminescent device using a compound 3 of Example 2.

FIG. 16 is a transient decay curve of the thin film organic photoluminescent device using the compound 3 of Example 2.

FIG. 17 is a light emission spectrum of an organic electroluminescent device using a compound 1 of Example 3.

FIG. 18 is a graph showing electric current density-voltage characteristics of the organic electroluminescent device using the compound 1 of Example 3.

FIG. 19 is a graph showing external quantum efficiency-electric current density characteristics of the organic electroluminescent device using the compound 1 of Example 3.

FIG. 20 is a light emission spectrum of an organic electroluminescent device using a compound 2 of Example 3.

FIG. 21 is a graph showing electric current density-voltage characteristics of the organic electroluminescent device using the compound 2 of Example 3.

FIG. 22 is a graph showing external quantum efficiency-electric current density characteristics of the organic electroluminescence device using the compound 2 of Example 3.

### DESCRIPTION OF EMBODIMENTS

The contents of the invention will be described in detail below. The constitutional elements may be described below with reference to representative embodiments and specific examples of the invention, but the invention is not limited to the embodiments and the examples. In the present specification, a numerical range expressed by “from X to Y” means a range including the numerals X and Y as the lower limit and the upper limit, respectively. In addition, isotopic species of a hydrogen atom in a molecule of a compound that is used in the invention are not particularly limited, and for example, all hydrogen atoms in a molecule may be ¹H, or a part or an entirety thereof may be ²h (deuterium D).

[Compound Represented by General Formula (1)]

The light emitting material of the invention is configured of a compound that has a structure represented by the following general formula (1):

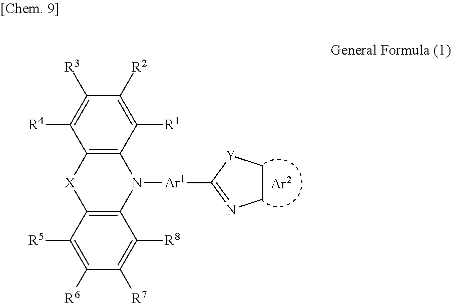

In the general formula (1), X represents O, S, N—R¹¹, C═O, C(R¹²)(R¹³), or Si(R¹⁴)(R¹⁵), and Y represents O, S, or N—R¹⁶. Ar¹ represents a substituted or unsubstituted arylene group, and Ar² represents an aromatic ring or a heteroaromatic ring. R¹ to R⁸ and R¹¹ to R¹⁶ each independently represent a hydrogen atom or a substituent, provided that R¹ and R², R² and R³, R³ and R⁴, R⁵ and R⁶, R⁶ and R⁷, R⁷ and R⁸ each may be bonded to each other to form a cyclic structure.

R¹ to R⁸ in the general formula (1) each independently represent a hydrogen atom or a substituent. All R¹ to R⁸ each may be a hydrogen atom. In the case where two or more thereof each are a substituent, the substituents may be the same as or different from each other. Examples of the substituent include a hydroxyl group, a halogen atom, a cyano group, an alkyl group having from 1 to 20 carbon atoms, an alkoxy group having from 1 to 20 carbon atoms, an alkylthio group having from 1 to 20 carbon atoms, an alkyl-substituted amino group having from 1 to 20 carbon atoms, an diaryl-substituted amino group having from 12 to 40 carbon atoms, an acyl group having from 2 to 20 carbon atoms, an aryl group having from 6 to 40 carbon atoms, a heteroaryl group having from 3 to 40 carbon atoms, a substituted or unsubstituted carbazolyl group having from 12 to 40 carbon atoms, an alkenyl group having from 2 to 10 carbon atoms, an alkynyl group having from 2 to 10 carbon atoms, an alkoxycarbonyl group having from 2 to 10 carbon atoms, an alkylsulfonyl group having from 1 to 10 carbon atoms, a haloalkyl group having from 1 to 10 carbon atoms, an amide group, an alkylamide group having from 2 to 10 carbon atoms, a trialkylsilyl group having from 3 to 20 carbon atoms, a trialkylsilylalkyl group having from 4 to 20 carbon atoms, a trialkylsilylalkenyl group having from 5 to 20 carbon atoms, a trialkylsilylalkynyl group having from 5 to 20 carbon atoms and a nitro group. Among these specific examples, the groups that may be further substituted with a substituent may be substituted. More preferred examples of the substituent include a halogen atom, a cyano group, a substituted or unsubstituted alkyl group having from 1 to 20 carbon atoms, an alkoxy group having from 1 to 20 carbon atoms, a substituted or unsubstituted aryl group having from 6 to 40 carbon atoms, a substituted or unsubstituted heteroaryl group having from 3 to 40 carbon atoms, a substituted or unsubstituted dialkylamino group having from 1 to 10 carbon atoms, a substituted or unsubstituted diarylamino group having from 12 to 40 carbon atoms and a substituted or unsubstituted carbazolyl group having from 12 to 40 carbon atoms. Further preferred examples of the substituent include a fluorine atom, a chlorine atom, a cyano group, a substituted or unsubstituted alkyl group having from 1 to 10 carbon atoms, a substituted or unsubstituted alkoxy group having from 1 to 10 carbon atoms, a substituted or unsubstituted dialkylamino group having from 1 to 10 carbon atoms, a substituted or unsubstituted diarylamino group having from 12 to 40 carbon atoms, a substituted or unsubstituted aryl group having from 6 to 15 carbon atoms and a substituted or unsubstituted heteroaryl group having from 3 to 12 carbon atoms.

The alkyl group referred herein may be any one of linear, branched and cyclic groups, and more preferably has from 1 to 6 carbon atoms, and specific examples thereof include a methyl group, an ethyl group, a propyl group, a butyl group, a tert-butyl group, a pentyl group, a hexyl group and an isopropyl group. The aryl group may be a monocyclic ring or a fused ring, and specific examples thereof include a phenyl group and a naphthyl group. The alkoxy group may be any one of linear, branched and cyclic groups, and more preferably has from 1 to 6 carbon atoms, and specific examples thereof include a methoxy group, an ethoxy group, a propoxy group, a butoxy group, a tert-butoxy group, a pentyloxy group, a hexyloxy group and isopropoxy group. The two alkyl groups of the dialkylamino group may be the same as or different from each other, and are preferably the same as each other. The two alkyl groups of the dialkylamino group each independently may be any one of linear, branched and cyclic groups, and more preferably has from 1 to 6 carbon atoms, and specific examples thereof include a methyl group, an ethyl group, a propyl group, a butyl group, a pentyl group, a hexyl group and an isopropyl group. The two alkyl groups of the dialkylamino group may be bonded to each other to form a cyclic structure with the nitrogen atom of the amino group. The aryl group that may be used as the substituent may be a monocyclic ring or a fused ring, and specific examples thereof include a phenyl group and a naphthyl group. The heteroaryl group may also be a monocyclic ring or a fused ring, and specific examples thereof include a pyridyl group, a pyridazyl group, a pyrimidyl group, a triazyl group, a triazolyl group and a benzotriazolyl group. The heteroaryl group may be a group that is bonded through the heteroatom or a group that is bonded through the carbon atom constituting the heteroaryl ring. The two aryl group of the diarylamino group each may be a monocyclic ring or a fused ring, and specific examples thereof include a phenyl group and a naphthyl group. The two aryl groups of the diarylamino group may be bonded to each other to form a cyclic structure with the nitrogen atom of the amino group, and specific examples thereof include a 9-carbazolyl group.

In the general formula (1), R¹ and R², R² and R³, R³ and R⁴, R⁵ and R⁶, R⁶ and R⁷, and R⁷ and R⁸ each may be bonded to each other to form a cyclic structure. The cyclic structure may be an aromatic ring or an aliphatic ring, and may be one containing a hetero atom. The hetero atom herein is preferably selected from the group consisting of a nitrogen atom, an oxygen atom and a sulfur atom. Examples of the cyclic structure formed include a benzene ring, a naphthalene ring, a pyridine ring, a pyridazine ring, a pyrimidine ring, a pyrazine ring, a pyrrole ring, an imidazole ring, a pyrazole ring, a triazole ring, an imidazoline ring, an oxazole ring, an isoxazole ring, a thiazole ring, an isothiazole ring, a cyclohexadiene ring, a cyclohexene ring, a cyclopentaene ring, a cycloheptatriene ring, a cycloheptadiene ring and a cycloheptaene ring.

X in the general formula (1) represents O, S, N—R¹¹, C═O, C(R¹²)(R¹³), or Si(R¹⁴)(R¹⁵), is preferably O, S, N—R¹¹, or C═O, and is more preferably O or S.

When X in the general formula (1) is N—R¹¹, R¹¹ represents a hydrogen atom or a substituent, and is preferably a substituent or unsubstituent alkyl group or a substituted or unsubstituted aryl group. The substituted or unsubstituted alkyl group preferably has from 1 to 20 carbon atoms, more preferably has from 1 to 10 carbon atoms, further preferably has 1 to 6 carbon atoms, and further preferably has from 1 to 3 carbon atoms. The substituted or unsubstituted aryl group preferably has from 6 to 20 carbon atoms, more preferably has from 6 to 14 carbon atoms, and further preferably has from 6 to 10 carbon atoms. The above description and preferable ranges of the substituents that are available for R¹ to R⁸ for substituents may be referenced for the alkyl group or the aryl group, and preferable examples include a substituted or unsubstituted alkyl group, a substituted or unsubstituted amino group, and a substituted or unsubstituted heteroaryl group. Specific examples of R¹¹ include a methyl group, an ethyl group, an n-propyl group, a phenyl group, a p-tolyl group, a diphenylaminophenyl group, a dinaphthylaminophenyl group, a triazinylphenyl group, and these groups that are further substituted with substituents (for example, an alkyl group having from 1 to 6 carbon atoms or an aryl group having from 6 to 10 carbon atoms).

When X in the general formula (1) is C(R¹²)(R¹³) or Si(R¹⁴)(R¹⁵), R¹² to R¹⁵ each represent a hydrogen atom or a substituent. The above description and preferable ranges of the substituents that are available for R¹ to R⁸ may be referenced for substituents for the substituent, and preferable examples include a substituted or unsubstituted alkyl group. The substituted or unsubstituted alkyl group preferably has from 1 to 20 carbon atoms, more preferably has from 1 to 10 carbon atoms, further preferably has from 1 to 6 carbon atoms, and still further preferably has from 1 to 3 carbon atoms. R¹² and R¹³ may be the same or may be different from each other, and R¹⁴ and R¹⁵ may be the same or may be different from each other. Preferably, R¹² and R¹³ are the same, and R¹⁴ and R¹⁵ are the same.

Although specific examples of C(R¹²)(R¹³) or Si(R¹⁴)(R¹⁵) include C(CH₃)₂, C(C₂H₅)₂, C(CH₃)(C₂H₅), C(C₃H₇)₂, Si(CH₃)₂, Si(C₂H₅)₂, Si(CH₃)(C₂H₅), and Si(C₃H₇)₂, specific examples of C(R¹²)(R¹³) or Si(R¹⁴)(R¹⁵) are not limited thereto.

Y in the general formula (1) represents O, S, or N—R¹⁶ and is preferably O or S.

When Y in the general formula (1) is N—R¹⁶, R¹⁶ represents a hydrogen atom or a substituent. The above description about R¹¹ may be referenced for preferable examples of R¹⁶.

Ar¹ in the general formula (1) represents a substituted or unsubstituted arylene group. The substituted or unsubstituted arylene group preferably has from 6 to 20 carbon atoms, more preferably has from 6 to 14 carbon atoms, and more preferably has from 6 to 10 carbon atoms. Although the above description and preferable ranges of the substituents that are available for R¹ to R⁸ may be referenced for substituents for the arylene group, and preferable examples include a substituted or unsubstituted alkyl group and a substituted or unsubstituted alkoxy group. The substituted or unsubstituted alkyl group and the substituted or unsubstituted alkoxy group described herein each preferably has from 1 to 10 carbon atoms, more preferably has from 1 to 6 carbon atoms, and further preferably has from 1 to 3 carbon atoms. Specific examples of Ar¹ include 1,4-phenylene group, 1,3-phenylene group, 1,4-naphthylene group, and 1,3-naphthylene group, and among them, 1,4-pheneylene group and 1,3-phenylene group are preferably used.

Ar² in the general formula (1) represents an aromatic ring or a heteroaromatic ring. Preferable examples of a hetero atom constituting a ring skeleton of the heteroaromatic ring include a nitrogen atom, and the number of hetero atoms constituting the ring skeleton is preferably from 1 to 3, and is more preferably 1 or 2. Specific examples of the aromatic ring or the heteroaromatic ring constituting Ar² include a benzene ring, pyridine ring, pyridazine ring, pyrimidine ring, and pyrazine ring. To the aromatic ring or the heteroaromatic ring constituting Ar², another ring structure may be fused. Examples of such a fused ring include an aromatic ring, a heteroaromatic ring, an aliphatic hydrocarbon ring, and a non-aromatic hetero ring. Preferable examples of a ring skeleton atom constituting such a fused ring include a carbon atom, a nitrogen atom, an oxygen atom, and a sulfur atom. In addition, such a fused ring is preferably a 5 to 7-membered ring and is more preferably a 5 or 6-membered ring.

The compound represented by the general formula (1) preferably contains a structure represented by the following general formula (2).

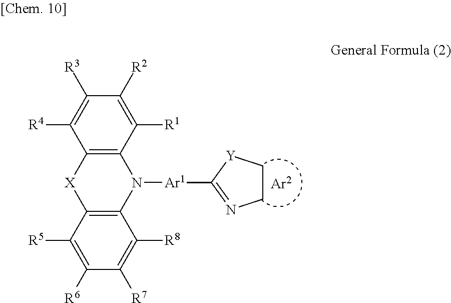

In the general formula (2), X represents O, S, N—R¹¹, C═O, C(R¹²)(R¹³), or Si(R¹⁴)(R¹⁵), and Y represents O, S, or N—R¹⁶. Ar² represents an aromatic ring or a heteroaromatic ring. R¹ to R⁸, R¹¹ to R¹⁶, and R²¹ to R²⁴ each independently represent a hydrogen atom or a substituent, provided that R¹ and R², R² and R³, R³ and R⁴, R⁵ and R⁶, R⁶ and R⁷, R⁷ and R⁸, R²¹ and R²², and R²³ and R²⁴ each may be bonded to each other to form a cyclic structure.

The above corresponding description and preferable ranges regarding the general formula (1) may be referenced for description and preferable ranges of X, Y, Ar², R¹ to R⁸, and R¹¹ to R¹⁶ in the general formula (2). In addition, the above description and preferable ranges of R¹ to R⁸ in the general formula (1) may be referenced for description and preferable ranges of R²¹ to R²⁴ in the general formula (2).

As R²¹ to R²⁴ in the general formula (2), a hydrogen atom, a substituted or unsubstituted alkyl group having from 1 to 10 carbon atoms, or a substituted or unsubstituted alkoxy group having from 1 to 10 carbon atoms is preferably used, a hydrogen atom, a substituted or unsubstituted alkyl group having from 1 to 6 carbon atoms, or a substituted or unsubstituted alkoxy group having from 1 to 6 carbon atoms is more preferably used, and a hydrogen atom, a substituted or unsubstituted alkyl group having from 1 to 3 carbon atoms, or a substituted or unsubstituted alkoxy group having from 1 to 3 carbon atoms is more preferably used. All R²¹ to R²⁴ may be hydrogen atoms or may be substituents. When two or more of R²¹ to R²⁴ are substituents, the two or more may be the same or different from each other. In addition, the corresponding description and preferable ranges of R¹ to R⁸ may be referenced for the ring structure that can be formed by R²¹ and R²² and R²³ and R²⁴ being bonded to each other.

The compound represented by the general formula (1) preferably contains a structure represented by the following general formula (3).

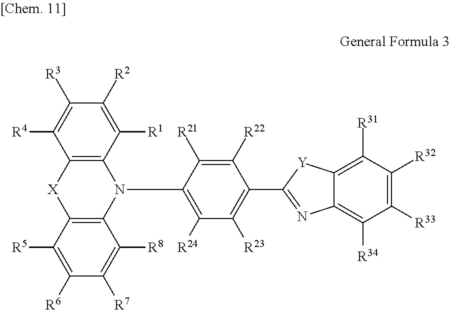

In the general formula (3), X represents O, S, N—R¹¹, C═O, C(R¹²)(R¹³), or Si(R¹⁴)(R¹⁵), and Y represents O, S, or N—R¹⁶. R¹ to R⁸, R¹¹ to R¹⁶, R²¹ to R²⁴, and R³¹ to R³⁴ each independently represent a hydrogen atom or a substituent, provided that R¹ and R², R² and R³, R³ and R⁴, R⁵ and R⁶, R⁶ and R⁷, R⁷ and R⁸, R²¹ and R²², R²³ and R²⁴, R³¹ and R³², R³² and R³³, and R³³ and R³⁴ each may be bonded to each other to form a cyclic structure.

The above corresponding description and preferable ranges regarding the general formulae (1) and (2) may be referenced for description and preferable ranges of X, Y, Ar^{e}, R¹ to R⁸, R¹¹ to R¹⁶ and R²¹ to R²⁴ in the general formula (3).

As R³¹ to R³⁴ in the general formula (3), a hydrogen atom, a substituted or unsubstituted alkyl group having from 1 to 10 carbon atoms, or a substituted or unsubstituted alkoxy group having from 1 to 10 carbon atoms is preferably used, a hydrogen atom, a substituted or unsubstituted alkyl group having from 1 to 6 carbon atoms, or a substituted or unsubstituted alkoxy group having from 1 to 6 carbon atoms is more preferably used. All R³¹ to R³⁴ may be hydrogen atoms or may be substituents. When two or more of R³¹ to R³⁴ are substituents, the two or more may be the same or different from each other. In addition, the corresponding description and preferable ranges of R¹ to R⁸ may be referenced for the ring structure that can be formed by R³¹ and R³², R³² and R³³, and R³³ and R³⁴ being bonded to each other.

Specific examples of the compound represented by the general formula (1) are shown below. However, the compound represented by the general formula (1) capable of being used in the invention is not construed as being limited to the specific examples.

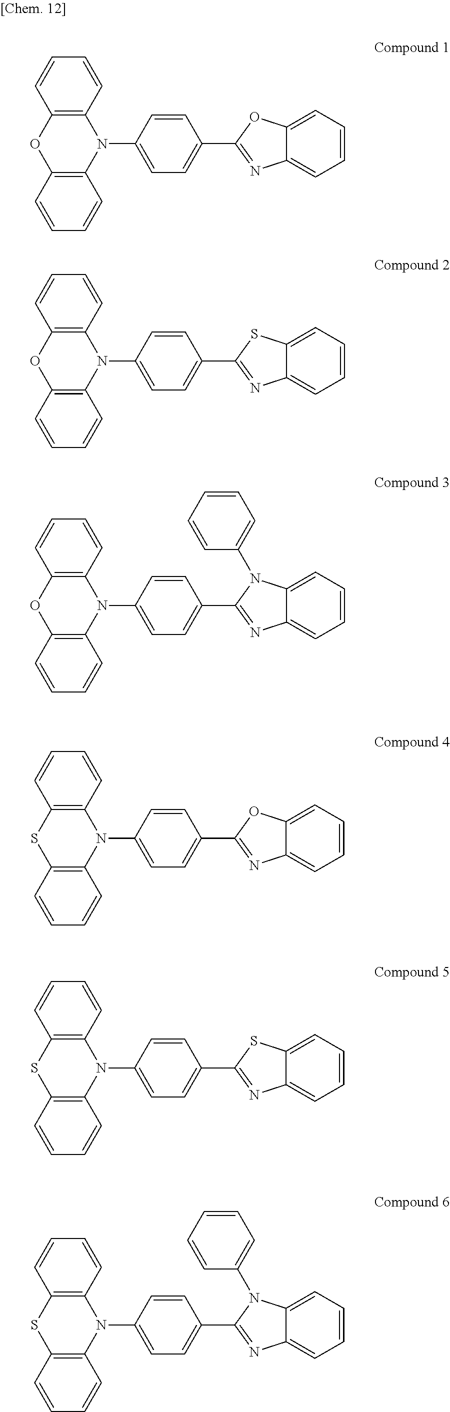

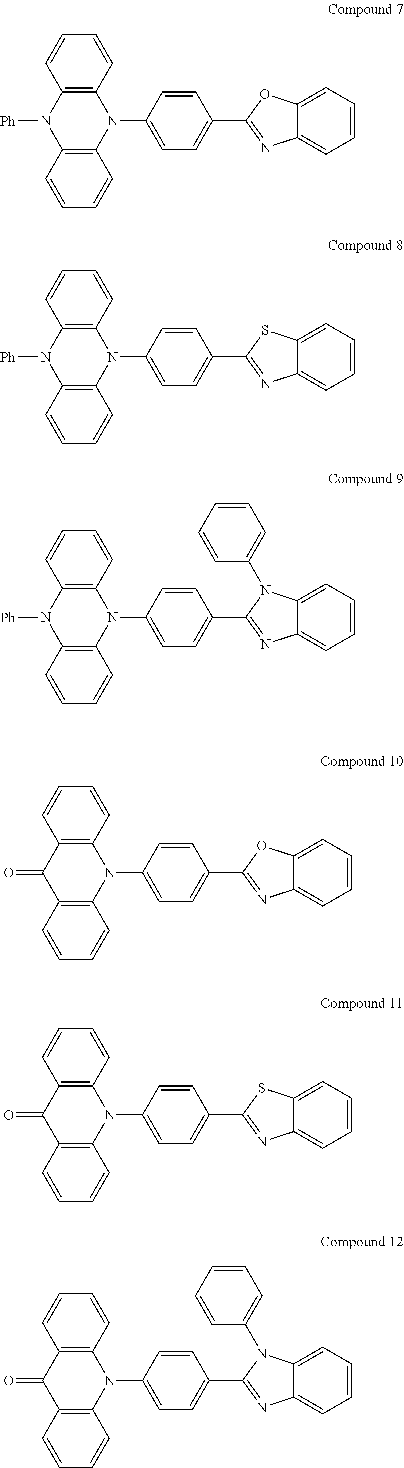

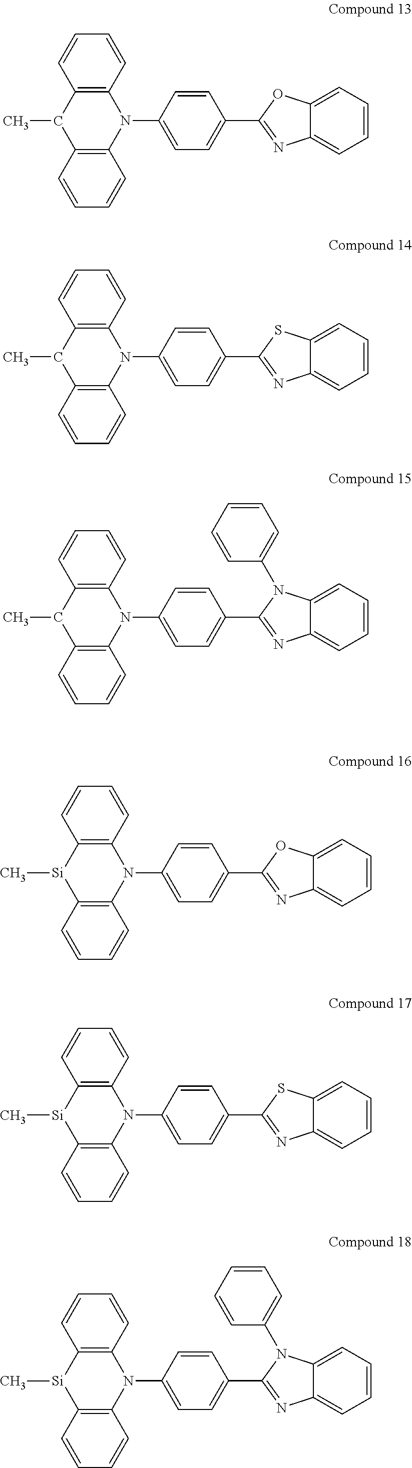

The molecular weight of the compound represented by the general formula (1) is preferably 1,500 or less, more preferably 1,200 or less, further preferably 1,000 or less, and still further preferably 800 or less, in the case where it is intended that an organic layer containing the compound represented by the general formula (1) is utilized by forming by a vapor deposition method. The lower limit of the molecular weight is the molecular weight of the smallest compound represented by the general formula (1).

The compound represented by the general formula (1) may be formed into a film by a coating method irrespective of the molecular weight thereof. The compound that has a relatively large molecular weight may be formed into a film by a coating method.

In an application embodiment of the invention, a compound that contains plural structures each represented by the general formula (1) in the molecule thereof may be used as a light emitting material.

For example, it is considered that a polymerizable group may be contained in advance in the structure represented by the general formula (1), and a polymer obtained by polymerizing the polymerizable group may be used as a light emitting material. Specifically, it is considered that a monomer that contains a polymerizable functional group in one of R¹ to R⁸, X, Y, Ar¹, and Ar² in the general formula (1) may be prepared, and may be homopolymerized or copolymerized with another monomer to prepare a polymer having repeating units, and the polymer may be used as a light emitting material. In alternative, it is also considered that the compounds each having the structure represented by the general formula (1) may be coupled to provide a dimer or a trimer, which may be used as a light emitting material.

Examples of the polymer having a repeating unit containing the structure represented by the general formula (1) include a polymer containing a structure represented by the following general formula (4) or (5):

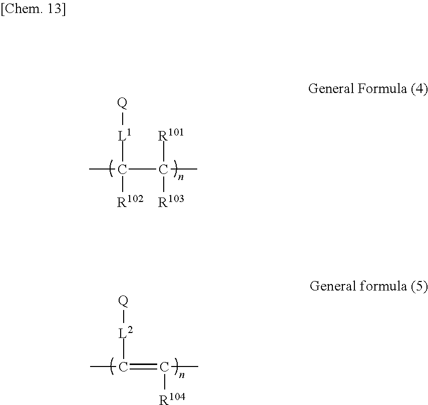

In the general formulae (4) and (5), Q represents a group containing the structure represented by the general formula (1), and L¹ and L² each represent a linking group. The linking group preferably has from 0 to 20 carbon atoms, more preferably from 1 to 15 carbon atoms, and further preferably from 2 to 10 carbon atoms. The linking group preferably has a structure represented by —X¹¹-L¹¹-, wherein X¹¹ represents an oxygen atom or a sulfur atom, and preferably represents an oxygen atom, and L¹¹ represents a linking group, preferably a substituted or unsubstituted alkylene group or a substituted or unsubstituted arylene group, and more preferably a substituted or unsubstituted alkylene group having from 1 to 10 carbon atoms or a substituted or unsubstituted phenylene group.

In the general formulae (4) and (5), R¹⁰¹, R¹⁰², R¹⁰³ and R¹⁰⁴ each independently represent a substituent, preferably a substituted or unsubstituted alkyl group having from 1 to 6 carbon atoms, a substituted or unsubstituted alkoxy group having from 1 to 6 carbon atoms or a halogen atom, more preferably an unsubstituted alkyl group having from 1 to 3 carbon atoms, an unsubstituted alkoxy group having from 1 to 3 carbon atoms, a fluorine atom or a chlorine atom, and further preferably an unsubstituted alkyl group having from 1 to 3 carbon atoms or an unsubstituted alkoxy group having from 1 to 3 carbon atoms.

The linking groups represented by L¹ and L² each are bonded to anyone of structures represented by the general formula (1) constituting the group represented by Q. Two or more of the linking groups may be bonded to one group represented by Q to form a crosslinked structure or a network structure.

Specific examples of the structure of the repeating unit include structures represented by the following formulae (6) to (9):

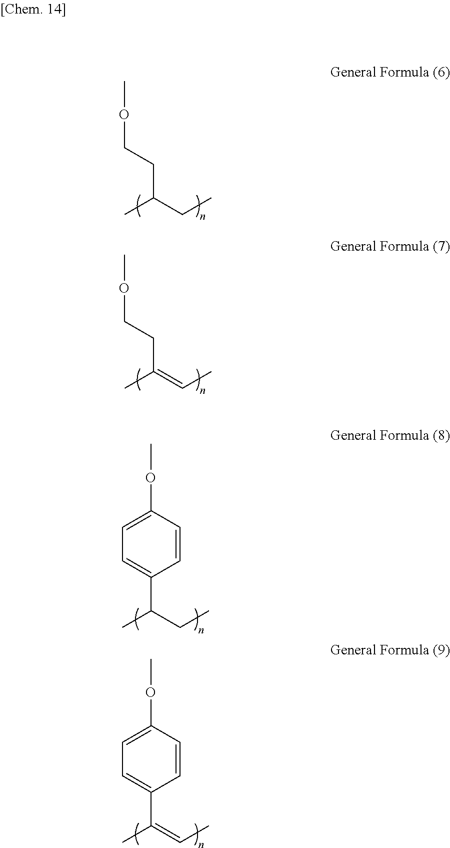

A polymer having a repeating unit containing a structure represented by the general formulae (6) to (9) may be synthesized in such a manner that a hydroxyl group is introduced as a part of the structure in the general formula (1), and the following compound is reacted with the hydroxyl group as a linker to introduce a polymerizable group, which is polymerized.

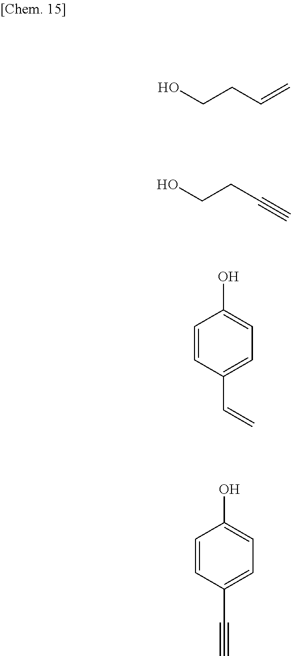

The polymer containing the structure represented by the general formula (1) in the molecule thereof may be a polymer that contains only a repeating unit having the structure represented by the general formula (1) or may be a polymer that contains another repeating unit in combination. One kind or two or more kinds of the repeating unit having the structure represented by the general formula (1) may be contained in the polymer. Examples of the repeating unit that does not have the structure represented by the general formula (1) include those derived from monomers that are used for ordinary copolymerization, and specific examples thereof include repeating units derived from monomers having an ethylenic unsaturated bond, such as ethylene and styrene. However, repeating units are not limited to the exemplified repeating units.

Among the compounds represented by the general formula (1), the compound represented by the following general formula (1′) is a new compound.

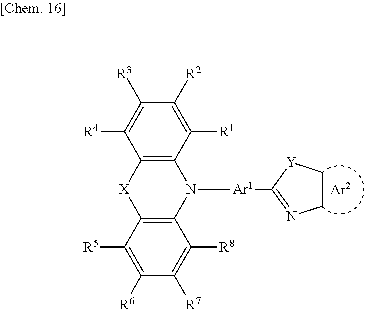

In the general formula (1′), X represents O, S, N—R¹¹, C═O, C(R¹²)(R¹³), or Si(R¹⁴)(R¹⁵), and Y represents O, S, or N—R¹⁶. Ar¹ represents a substituted or unsubstituted arylene group, and Ar² represents an aromatic ring or a heteroaromatic ring. R¹ to R⁸ and R¹¹ to R¹⁶ each independently represent a hydrogen atom or a substituent, provided that R¹⁶ is not a phenyl group when X is O, and R¹ and R², R² and R³, R³ and R⁴, R⁵ and R⁶, R⁶ and R⁷, R⁷ and R⁸ each may be bonded to each other to form a cyclic structure.

The corresponding description and reference ranges of the general formula (1′) may be referenced for the description and preferable ranges of X, Y, Ar¹, Ar², R¹ to R⁸, and R¹¹ to R¹⁶ in the general formula (1′).

[Synthesis Method of Compound Represented by General Formula (1)]

The compound represented by the general formula (1) may be synthesized by a combination of known reactions. For example, the synthesis may be performed by causing a compound represented by the following general formula (11) to react with a compound represented by the following general formula (12) according the following scheme. The reaction itself is a known reaction, and known reaction conditions may be appropriately selected. The compound represented by the general formula (12) may be synthesized, for example, by a corresponding chloride is converted to an amine and then further converted to a bromide.

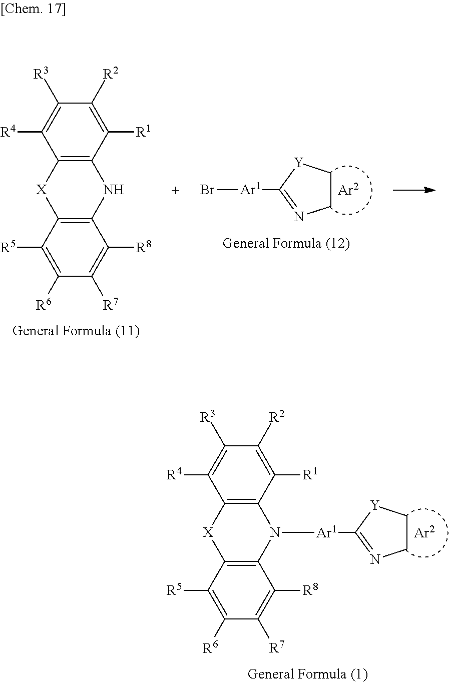

For the definitions for X, Y, Ar¹, Ar², and R¹ to R⁸ in the scheme, corresponding descriptions in the general formula (1) may be referenced.

For the details of the reaction, synthesis examples described later may be referenced. The compound represented by the general formula (1) may be synthesized by a combination of other known synthesis reactions.

[Organic Light Emitting Device]

The compound represented by the general formula (1) of the invention is useful as a light emitting material of an organic light emitting device. Accordingly, the compound represented by the general formula (1) of the invention may be effectively used as a light emitting material in a light emitting layer of an organic light emitting device. The compound represented by the general formula (1) includes a delayed fluorescent emitter emitting delayed fluorescent light. An organic light emitting device that uses the compound as a light emitting material thus has features that the device emits delayed fluorescent light and has a high light emission efficiency. The principle of the features may be described as follows for an organic electroluminescent device as an example.

In an organic electroluminescent device, carriers are injected from an anode and a cathode to a light emitting material to form an excited state for the light emitting material, with which light is emitted. In the case of a carrier injection type organic electroluminescent device, in general, excitons that are excited to the excited singlet state are 25% of the total excitons generated, and the remaining 75% thereof are excited to the excited triplet state. Accordingly, the use of phosphorescence, which is light emission from the excited triplet state, provides a high energy use efficiency. However, the excited triplet state has a long lifetime and thus causes saturation of the excited state and deactivation of energy through mutual action with the excitons in the excited triplet state, and therefore the quantum yield of phosphorescence may generally be often not high. A delayed fluorescent emitter emits fluorescent light through the mechanism that the energy of excitons transits to the excited triplet state through intersystem crossing or the like, and then transits to the excited singlet state through reverse intersystem crossing due to triplet-triplet annihilation or absorption of thermal energy, thereby emitting fluorescent light. It is considered that among the materials, a thermal activation type delayed fluorescent emitter emitting light through absorption of thermal energy is particularly useful for an organic electroluminescent device. In the case where a delayed fluorescent emitter is used in an organic electroluminescent device, the excitons in the excited singlet state normally emit fluorescent light. On the other hand, the excitons in the excited triplet state emit fluorescent light through intersystem crossing to the excited singlet state by absorbing the heat generated by the device. At this time, the light emitted through reverse intersystem crossing from the excited triplet state to the excited single state has the same wavelength as fluorescent light since it is light emission from the excited single state, but has a longer lifetime (light emission lifetime) than the normal fluorescent light and phosphorescent light, and thus the light is observed as fluorescent light that is delayed from the normal fluorescent light and phosphorescent light. The light may be defined as delayed fluorescent light. The use of the thermal activation type exciton transition mechanism may raise the proportion of the compound in the excited single state, which is generally formed in a proportion only of 25%, to 25% or more through the absorption of the thermal energy after the carrier injection. A compound that emits strong fluorescent light and delayed fluorescent light at a low temperature of lower than 100° C. undergoes the intersystem crossing from the excited triplet state to the excited singlet state sufficiently with the heat of the device, thereby emitting delayed fluorescent light, and thus the use of the compound may drastically enhance the light emission efficiency.

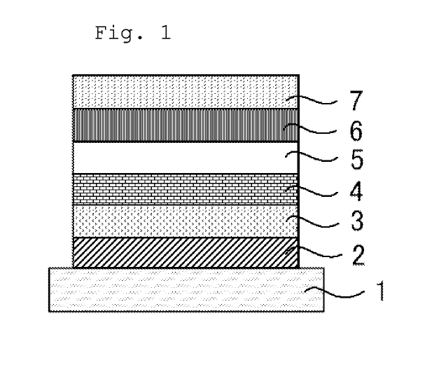

The use of the compound represented by the general formula (1) of the invention as a light emitting material of a light emitting layer may provide an excellent organic light emitting device, such as an organic photoluminescent device (organic PL device) and an organic electroluminescent device (organic EL device). The organic photoluminescent device has a structure containing a substrate having formed thereon at least a light emitting layer. The organic electroluminescent device has a structure containing at least an anode, a cathode and an organic layer formed between the anode and the cathode. The organic layer contains at least a light emitting layer, and may be formed only of a light emitting layer, or may have one or more organic layer in addition to the light emitting layer. Examples of the organic layer include a hole transporting layer, a hole injection layer, an electron barrier layer, a hole barrier layer, an electron injection layer, an electron transporting layer and an exciton barrier layer. The hole transporting layer may be a hole injection and transporting layer having a hole injection function, and the electron transporting layer may be an electron injection and transporting layer having an electron injection function. A specific structural example of an organic electroluminescent device is shown in FIG. 1. In FIG. 1, the numeral 1 denotes a substrate, 2 denotes an anode, 3 denotes a hole injection layer, 4 denotes a hole transporting layer, 5 denotes a light emitting layer, 6 denotes an electron transporting layer, and 7 denotes a cathode.

The members and the layers of the organic electroluminescent device will be described below. The descriptions for the substrate and the light emitting layer may also be applied to the substrate and the light emitting layer of the organic photoluminescent device.

(Substrate)

The organic electroluminescent device of the invention is preferably supported by a substrate. The substrate is not particularly limited and may be those that have been commonly used in an organic electroluminescent device, and examples thereof used include those formed of glass, transparent plastics, quartz and silicon.

(Anode)

The anode of the organic electroluminescent device used is preferably formed of as an electrode material a metal, an alloy or an electroconductive compound each having a large work function (4 eV or more), or a mixture thereof. Specific examples of the electrode material include a metal, such as Au, and an electroconductive transparent material, such as CuI, indium tin oxide (ITO), SnO₂ and ZnO. A material that is amorphous and is capable of forming a transparent electroconductive film, such as IDIXO (In₂O₃—ZnO), may also be used. The anode may be formed in such a manner that the electrode material is formed into a thin film by such a method as vapor deposition or sputtering, and the film is patterned into a desired pattern by a photolithography method, or in the case where the pattern may not require high accuracy (for example, approximately 100 μm or more), the pattern may be formed with a mask having a desired shape on vapor deposition or sputtering of the electrode material. In alternative, in the case where a material capable of being applied as a coating, such as an organic electroconductive compound, is used, a wet film forming method, such as a printing method and a coating method, may be used. In the case where emitted light is to be taken out through the anode, the anode preferably has a transmittance of more than 10%, and the anode preferably has a sheet resistance of several hundred ohm per square or less. The thickness thereof may be generally selected from a range of from 10 to 1,000 nm, and preferably from 10 to 200 nm, while depending on the material used.

(Cathode)

The cathode is preferably formed of as an electrode material a metal having a small work function (4 eV or less) (referred to as an electron injection metal), an alloy or an electroconductive compound each having a small work function (4 eV or less), or a mixture thereof. Specific examples of the electrode material include sodium, a sodium-potassium alloy, magnesium, lithium, a magnesium-cupper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth metal. Among these, a mixture of an electron injection metal and a second metal that is a stable metal having a larger work function than the electron injection metal, for example, a magnesium-silver mixture, a magnesium-aluminum mixture, a magnesium-indium mixture, an aluminum-aluminum oxide (Al₂O₃) mixture, a lithium-aluminum mixture, and aluminum, are preferred from the standpoint of the electron injection property and the durability against oxidation and the like. The cathode may be produced by forming the electrode material into a thin film by such a method as vapor deposition or sputtering. The cathode preferably has a sheet resistance of several hundred ohm per square or less, and the thickness thereof may be generally selected from a range of from 10 nm to 5 μm, and preferably from 50 to 200 nm. For transmitting the emitted light, any one of the anode and the cathode of the organic electroluminescent device is preferably transparent or translucent, thereby enhancing the light emission luminance.

The cathode may be formed with the electroconductive transparent materials described for the anode, thereby forming a transparent or translucent cathode, and by applying the cathode, a device having an anode and a cathode, both of which have transmittance, may be produced.

(Light Emitting Layer)

The light emitting layer is a layer, in which holes and electrons injected from the anode and the cathode, respectively, are recombined to form excitons, and then the layer emits light. A light emitting material may be solely used as the light emitting layer, but the light emitting layer preferably contains a light emitting material and a host material. The light emitting material used may be one kind or two or more kinds selected from the group of compounds represented by the general formula (1) of the invention. In order that the organic electroluminescent device and the organic photoluminescent device of the invention exhibit a high light emission efficiency, it is important that the singlet excitons and the triplet excitons generated in the light emitting material are confined in the light emitting material. Accordingly, a host material is preferably used in addition to the light emitting material in the light emitting layer. The host material used may be an organic compound that has excited singlet energy and excited triplet energy, at least one of which is higher than those of the light emitting material of the invention. As a result, the singlet excitons and the triplet excitons generated in the light emitting material of the invention are capable of being confined in the molecules of the light emitting material of the invention, thereby eliciting the light emission efficiency thereof sufficiently. However, there are cases where a high light emission efficiency is obtained even though the singlet excitons and the triplet excitons may not be sufficiently confined, and therefore host materials capable of achieving a high light emission efficiency may be used in the invention without any particular limitation. In the organic light emitting device and the organic electroluminescent device of the invention, the light emission occurs in the light emitting material of the invention contained in the light emitting layer. The emitted light contains both fluorescent light and delayed fluorescent light. However, a part of the emitted light may contain emitted light from the host material, or the emitted light may partially contain emitted light from the host material.

In the case where the host material is used, the amount of the compound of the invention as the light emitting material contained in the light emitting layer is preferably 0.1% by weight or more, and more preferably 1% by weight or more, and is preferably 50% by weight or less, more preferably 20% by weight or less, and further preferably 10% by weight or less.

The host material in the light emitting layer is preferably an organic compound that has a hole transporting function and an electron transporting function, prevents the emitted light from being increased in wavelength, and has a high glass transition temperature.

(Injection Layer)

The injection layer is a layer that is provided between the electrode and the organic layer for decreasing the driving voltage and enhancing the light emission luminance, and includes a hole injection layer and an electron injection layer, which may be provided between the anode and the light emitting layer or the hole transporting layer and between the cathode and the light emitting layer or the electron transporting layer. The injection layer may be provided depending on necessity.

(Barrier Layer)

The barrier layer is a layer that is capable of inhibiting charges (electrons or holes) and/or excitons present in the light emitting layer from being diffused outside the light emitting layer. The electron barrier layer may be disposed between the light emitting layer and the hole transporting layer, and inhibits electrons from passing through the light emitting layer toward the hole transporting layer. Similarly, the hole barrier layer may be disposed between the light emitting layer and the electron transporting layer, and inhibits holes from passing through the light emitting layer toward the electron transporting layer. The barrier layer may also be used for inhibiting excitons from being diffused outside the light emitting layer. Thus, the electron barrier layer and the hole barrier layer each may also have a function as an exciton barrier layer. The electron barrier layer or the exciton barrier layer referred herein means a layer that has both the functions of an electron barrier layer and an exciton barrier layer by one layer.

(Hole Barrier Layer)

The hole barrier layer has the function of an electron transporting layer in a broad sense. The hole barrier layer has a function of inhibiting holes from reaching the electron transporting layer while transporting electrons, and thereby enhances the recombination probability of electrons and holes in the light emitting layer. As the material for the hole barrier layer, the materials for the electron transporting layer described later may be used depending on necessity.

(Electron Barrier Layer)

The electron barrier layer has the function of transporting holes in a broad sense. The electron barrier layer has a function of inhibiting electrons from reaching the hole transporting layer while transporting holes, and thereby enhances the recombination probability of electrons and holes in the light emitting layer.

(Exciton Barrier Layer)

The exciton barrier layer is a layer for inhibiting excitons generated through recombination of holes and electrons in the light emitting layer from being diffused to the charge transporting layer, and the use of the layer inserted enables effective confinement of excitons in the light emitting layer, and thereby enhances the light emission efficiency of the device. The exciton barrier layer may be inserted adjacent to the light emitting layer on any of the side of the anode and the side of the cathode, and on both the sides. Specifically, in the case where the exciton barrier layer is present on the side of the anode, the layer may be inserted between the hole transporting layer and the light emitting layer and adjacent to the light emitting layer, and in the case where the layer is inserted on the side of the cathode, the layer may be inserted between the light emitting layer and the cathode and adjacent to the light emitting layer. Between the anode and the exciton barrier layer that is adjacent to the light emitting layer on the side of the anode, a hole injection layer, an electron barrier layer and the like may be provided, and between the cathode and the exciton barrier layer that is adjacent to the light emitting layer on the side of the cathode, an electron injection layer, an electron transporting layer, a hole barrier layer and the like may be provided. In the case where the barrier layer is provided, the material used for the barrier layer preferably has excited singlet energy and excited triplet energy, at least one of which is higher than the excited singlet energy and the excited triplet energy of the light emitting layer, respectively.

(Hole Transporting Layer)

The hole transporting layer is formed of a hole transporting material having a function of transporting holes, and the hole transporting layer may be provided as a single layer or plural layers.

The hole transporting material has one of injection or transporting property of holes and barrier property of electrons, and may be any of an organic material and an inorganic material. Examples of known hole transporting materials that may be used herein include a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer, particularly a thiophene oligomer. Among these, a porphyrin compound, an aromatic tertiary amine compound and a styrylamine compound are preferably used, and an aromatic tertiary amine compound is more preferably used.

(Electron Transporting Layer)

The electron transporting layer is formed of a material having a function of transporting electrons, and the electron transporting layer may be provided as a single layer or plural layers.

The electron transporting material (which may also function as a hole barrier material in some cases) may have a function of transporting electrons, which are injected from the cathode, to the light emitting layer. Examples of the electron transporting layer that may be used herein include a nitro-substituted fluorene derivative, a diphenylquinone derivative, a thiopyran dioxide derivative, carbodiimide, a fluorenylidene methane derivative, anthraquinodimethane and anthrone derivatives, and an oxadiazole derivative. The electron transporting material used may be a thiadiazole derivative obtained by replacing the oxygen atom of the oxadiazole ring of the oxadiazole derivative by a sulfur atom, or a quinoxaline derivative having a quinoxaline ring, which is known as an electron attracting group. Furthermore, polymer materials having these materials introduced to the polymer chain or having these materials used as the main chain of the polymer may also be used.

In the production of the organic electroluminescent device, the compound represented by the general formula (1) may be used not only in the light emitting layer but also in the other layers than the light emitting layer. In this case, the compound represented by the general formula (1) used in the light emitting layer and the compound represented by the general formula (1) used in the other layers than the light emitting layer may be the same as or different from each other. For example, the compound represented by the general formula (1) may be used in the injection layer, the barrier layer, the hole barrier layer, the electron barrier layer, the exciton barrier layer, the hole transporting layer, the electron transporting layer and the like described above. The film forming method of the layers are not particularly limited, and the layers may be produced by any of a dry process and a wet process.

Specific examples of preferred materials that may be used in the organic electroluminescent device are shown below, but the materials that may be used in the invention are not construed as being limited to the example compounds. The compound that is shown as a material having a particular function may also be used as a material having another function. In the structural formulae of the example compounds, R and R₁ to R₁₀ each independently represent a hydrogen atom or a substituent; n represents an integer of from 3 to 5.

Preferred examples of a compound that may also be used as the host material of the light emitting layer are shown below.

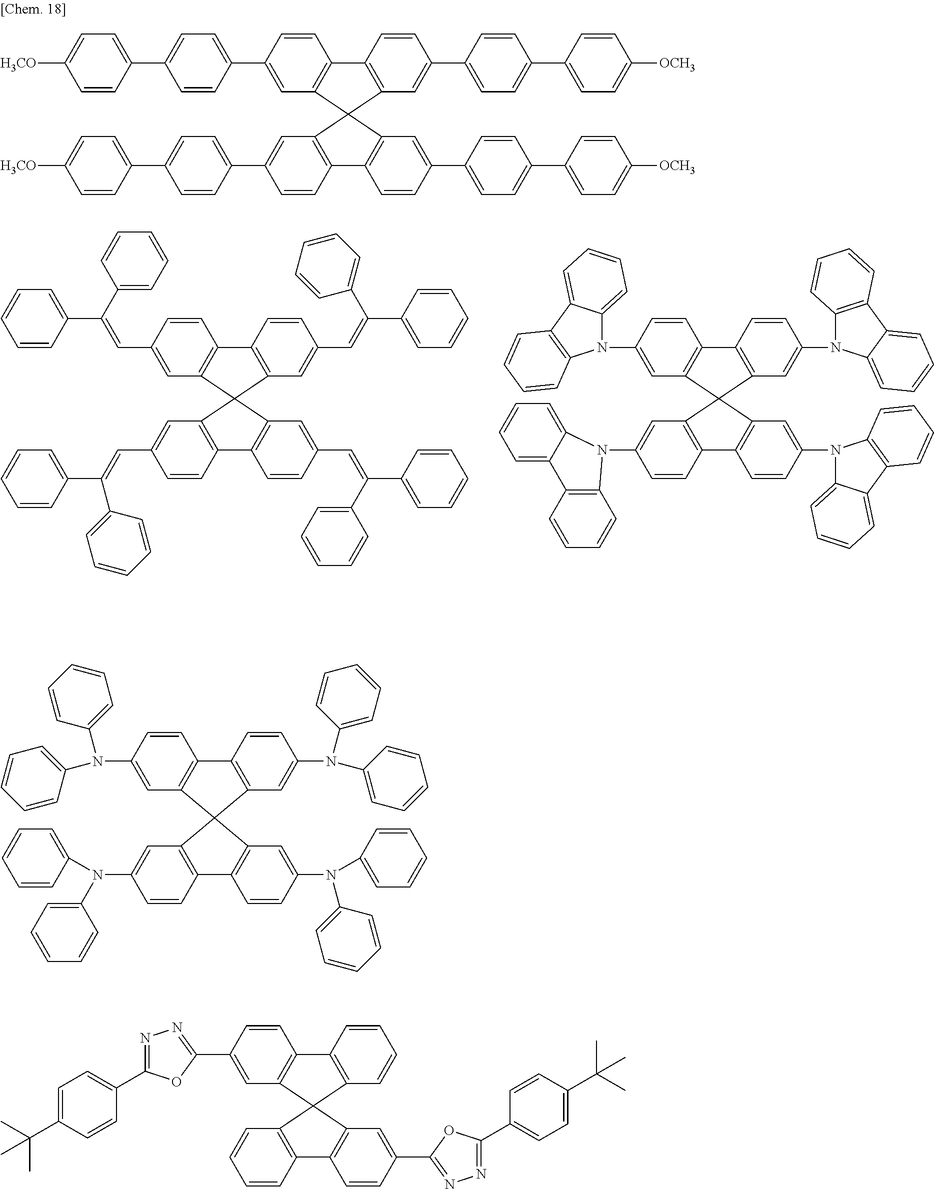

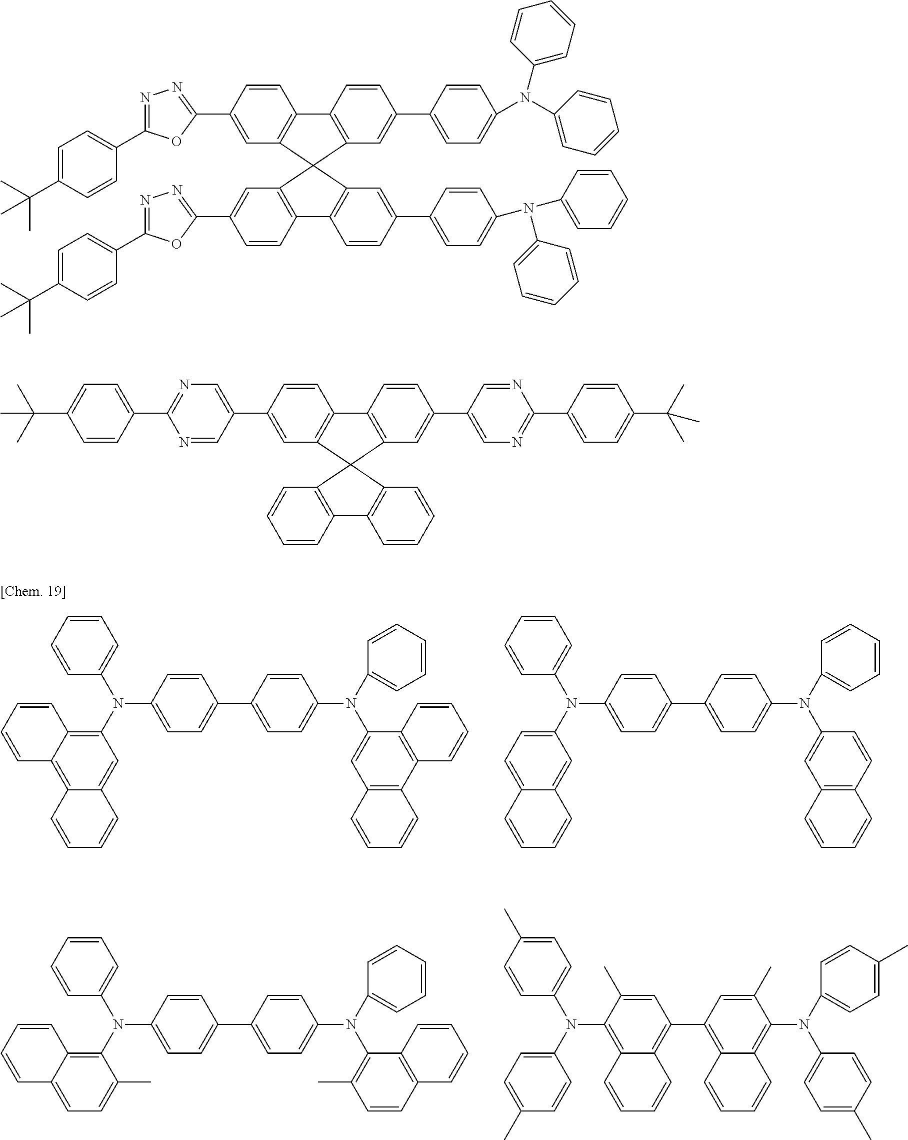

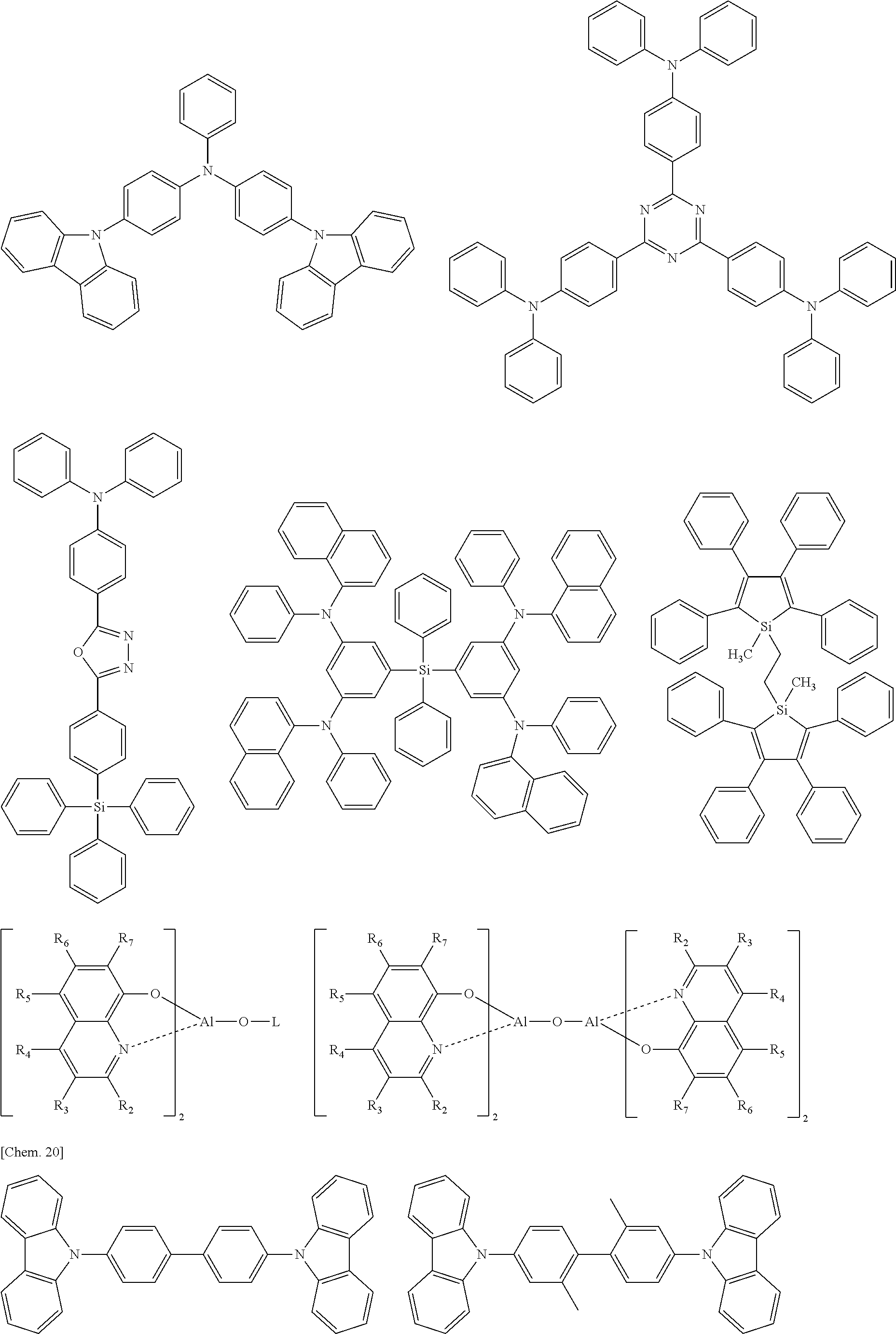

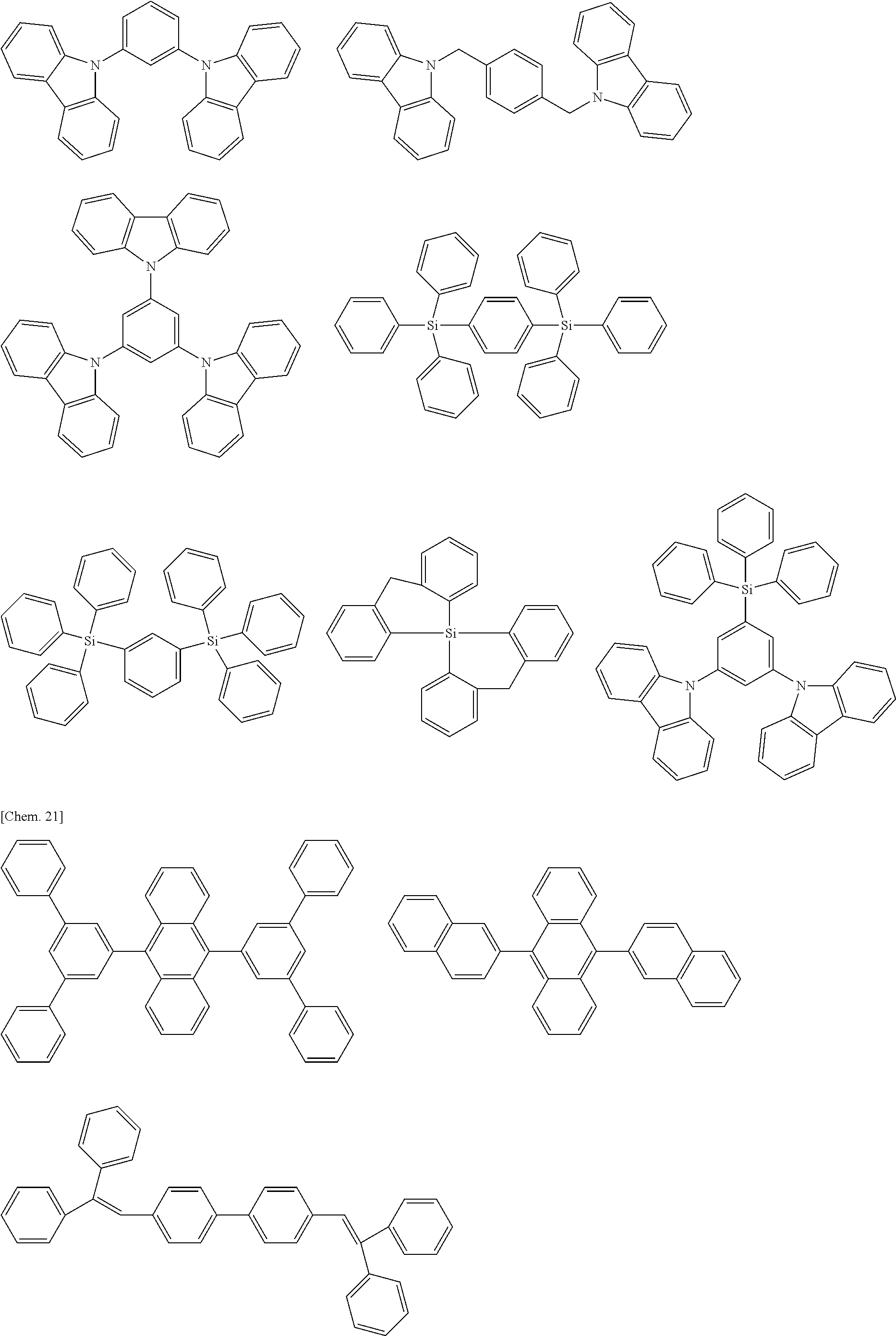

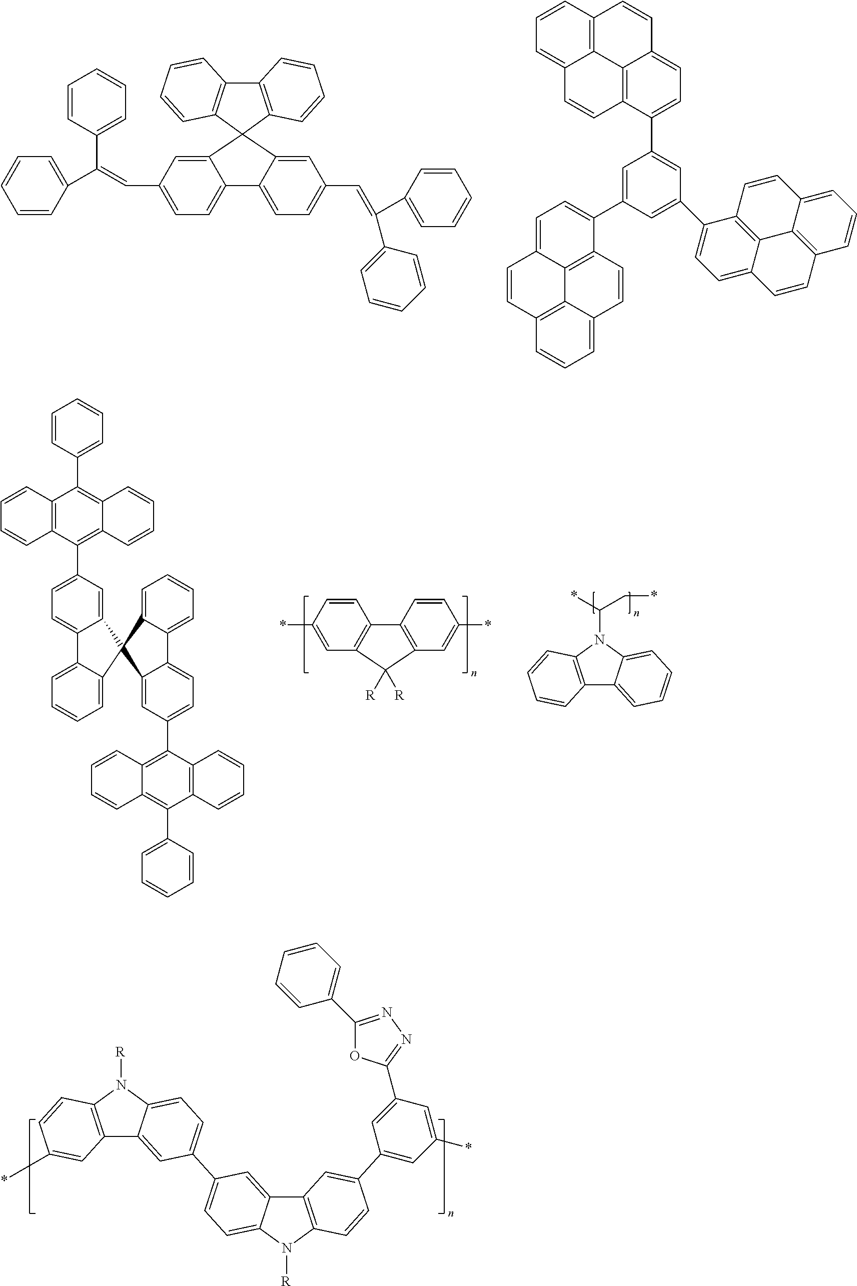

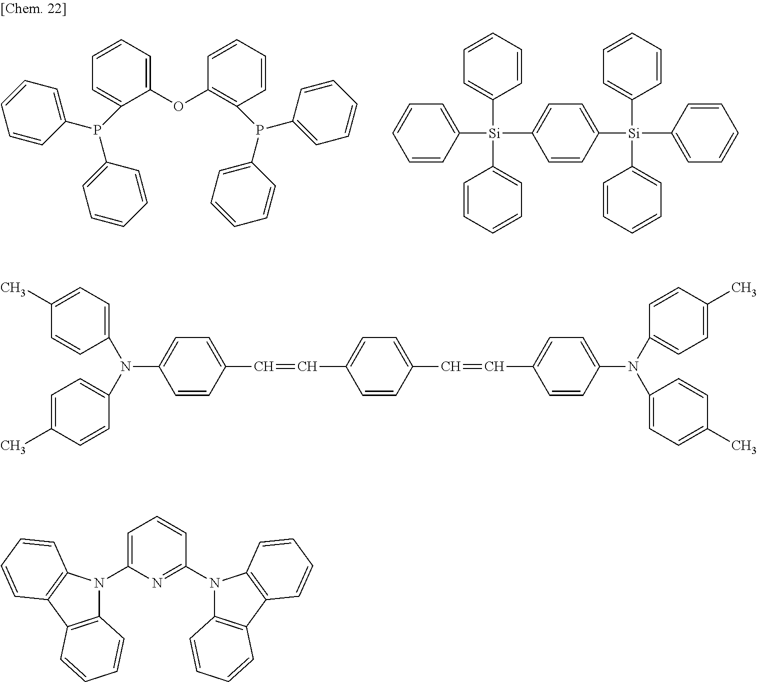

Preferred examples of a compound that may be used as the hole injection material are shown below.

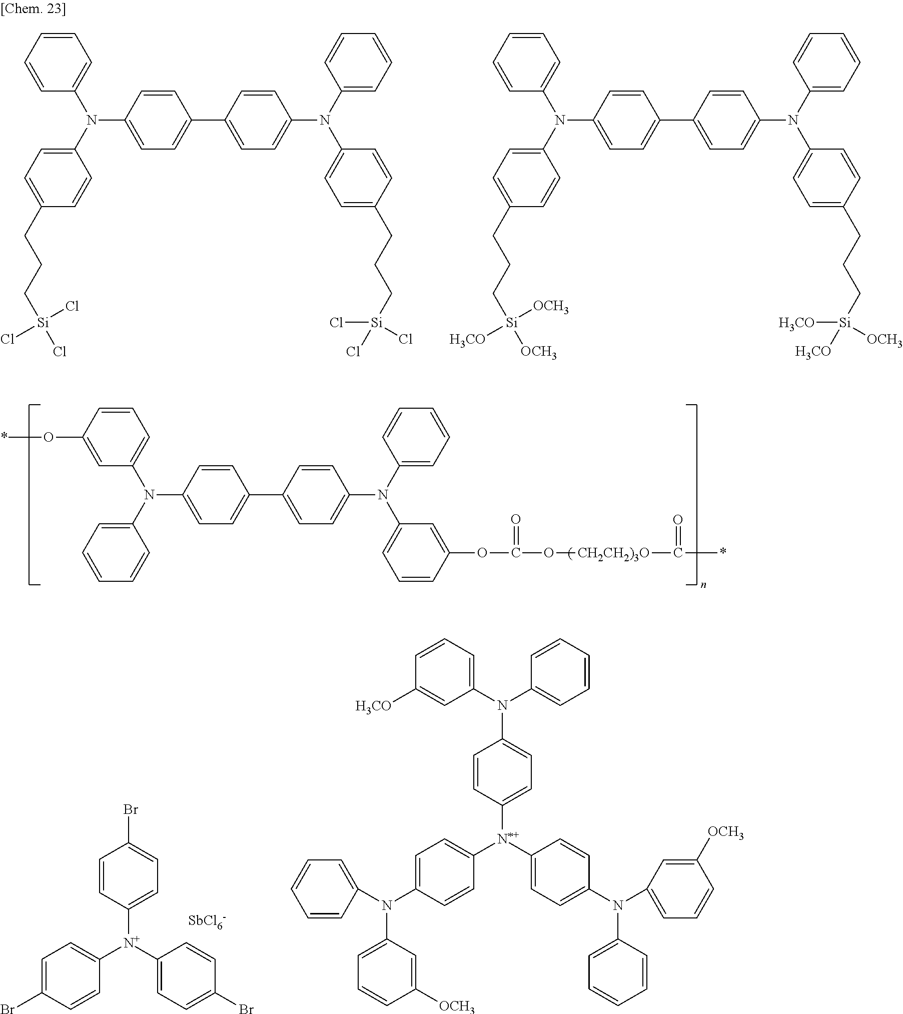

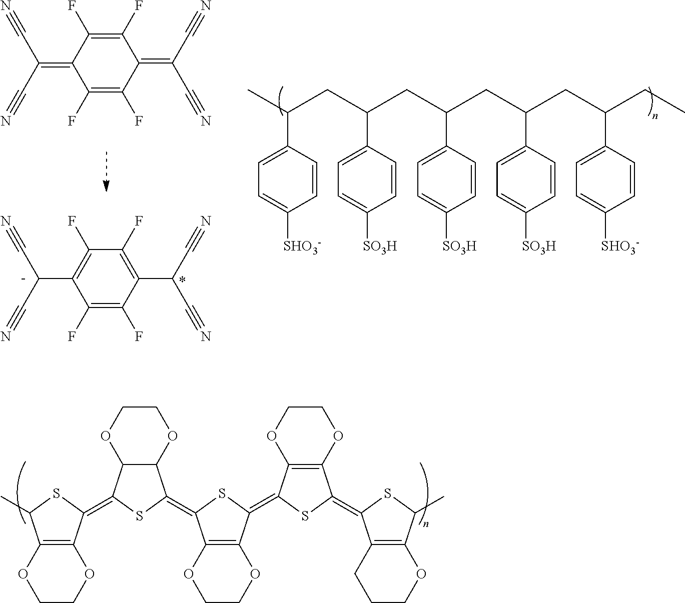

Preferred examples of a compound that may be used as the hole transporting material are shown below.

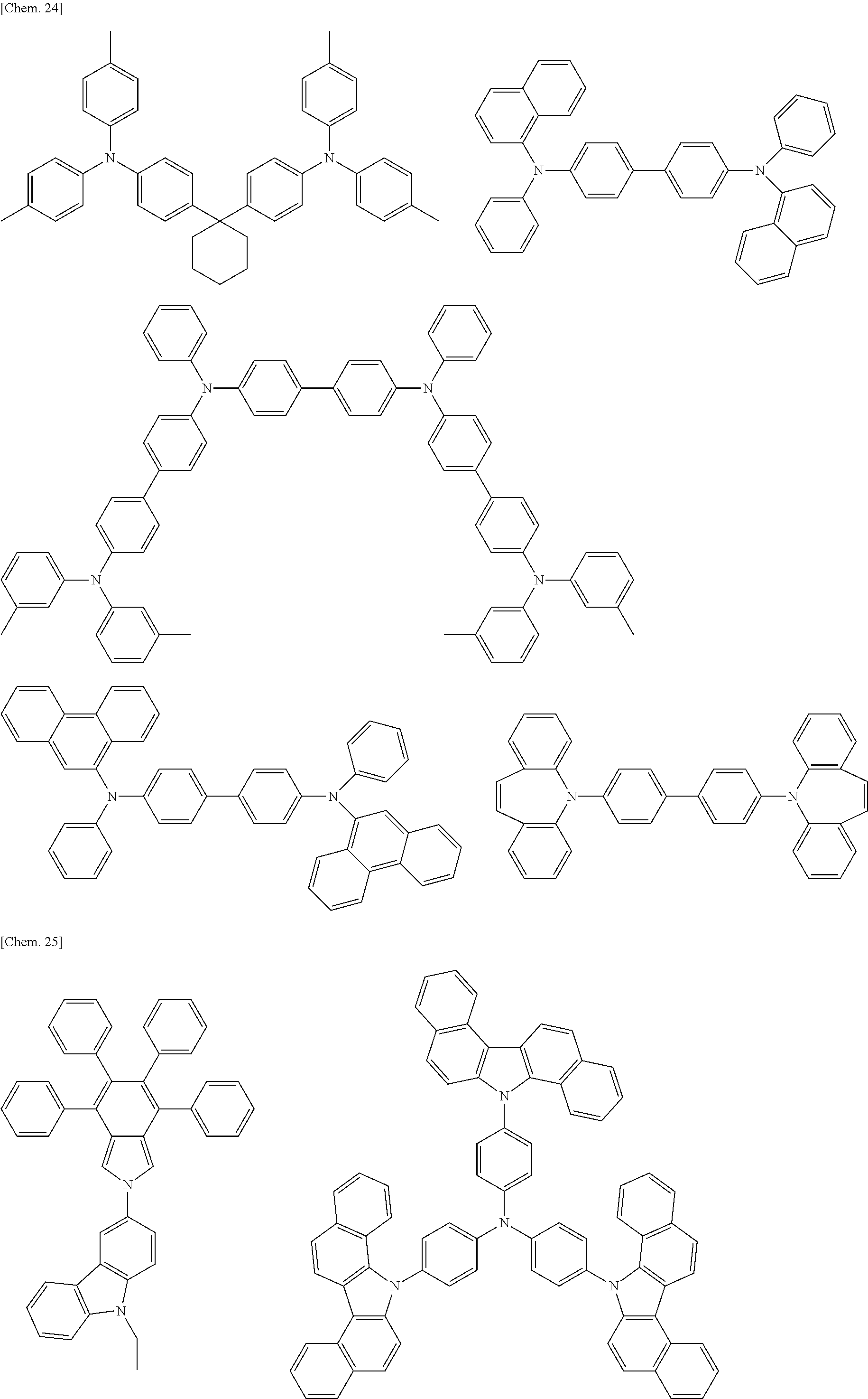

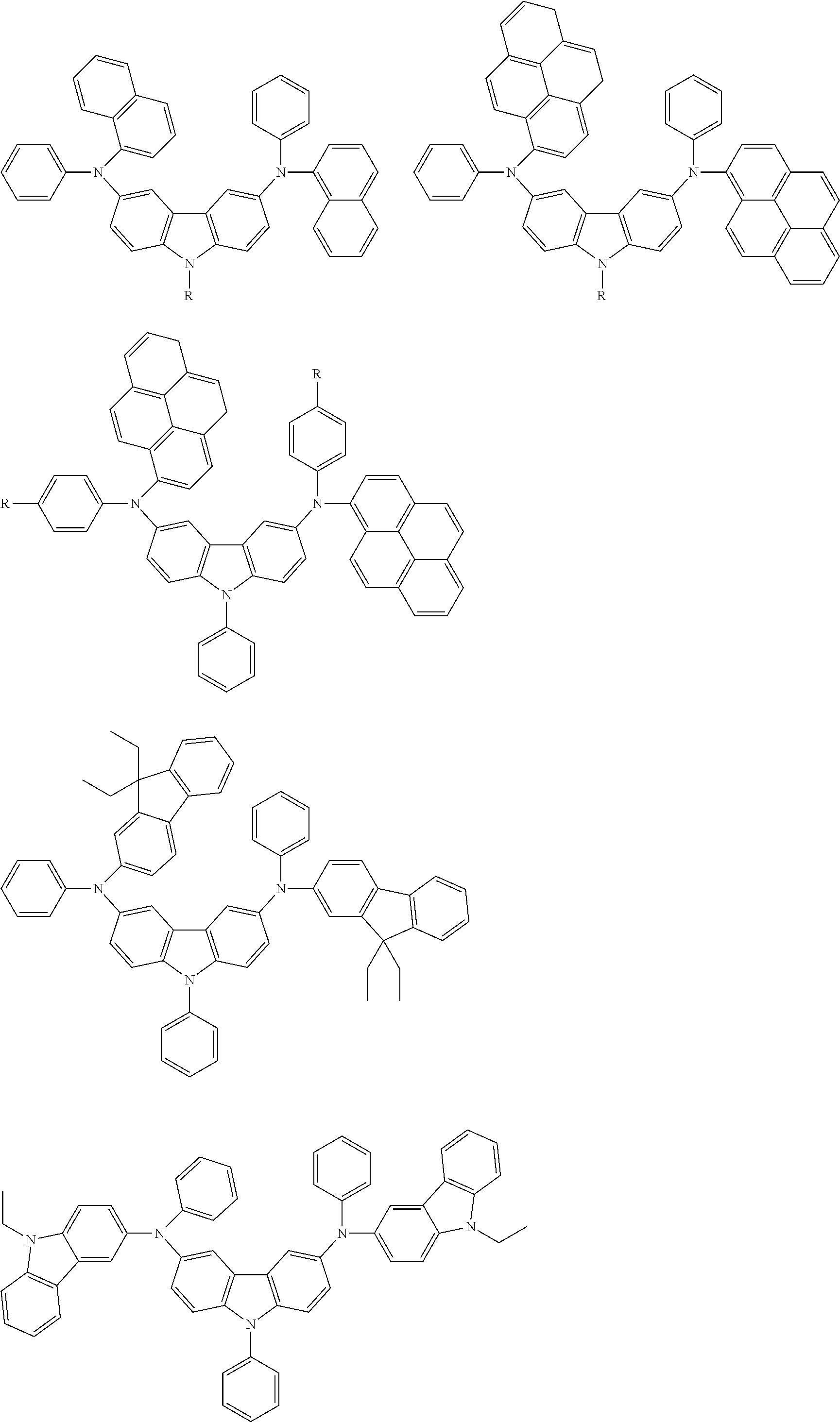

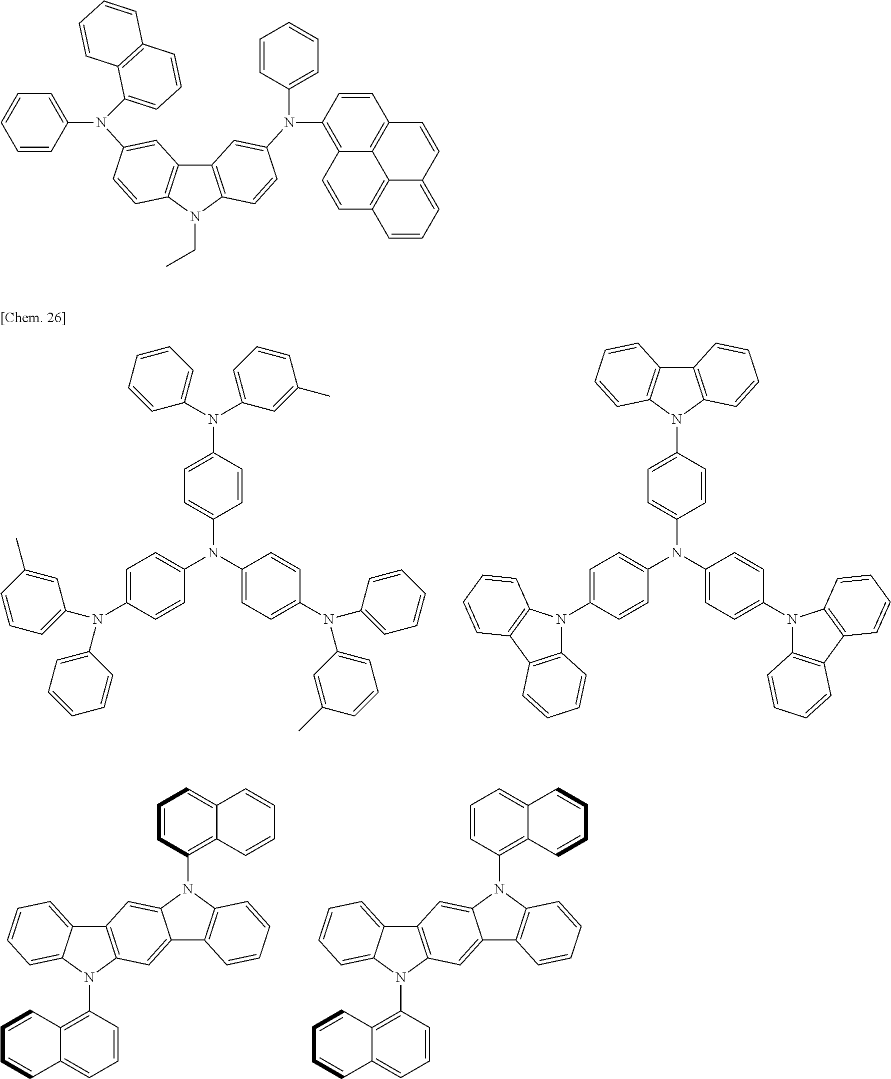

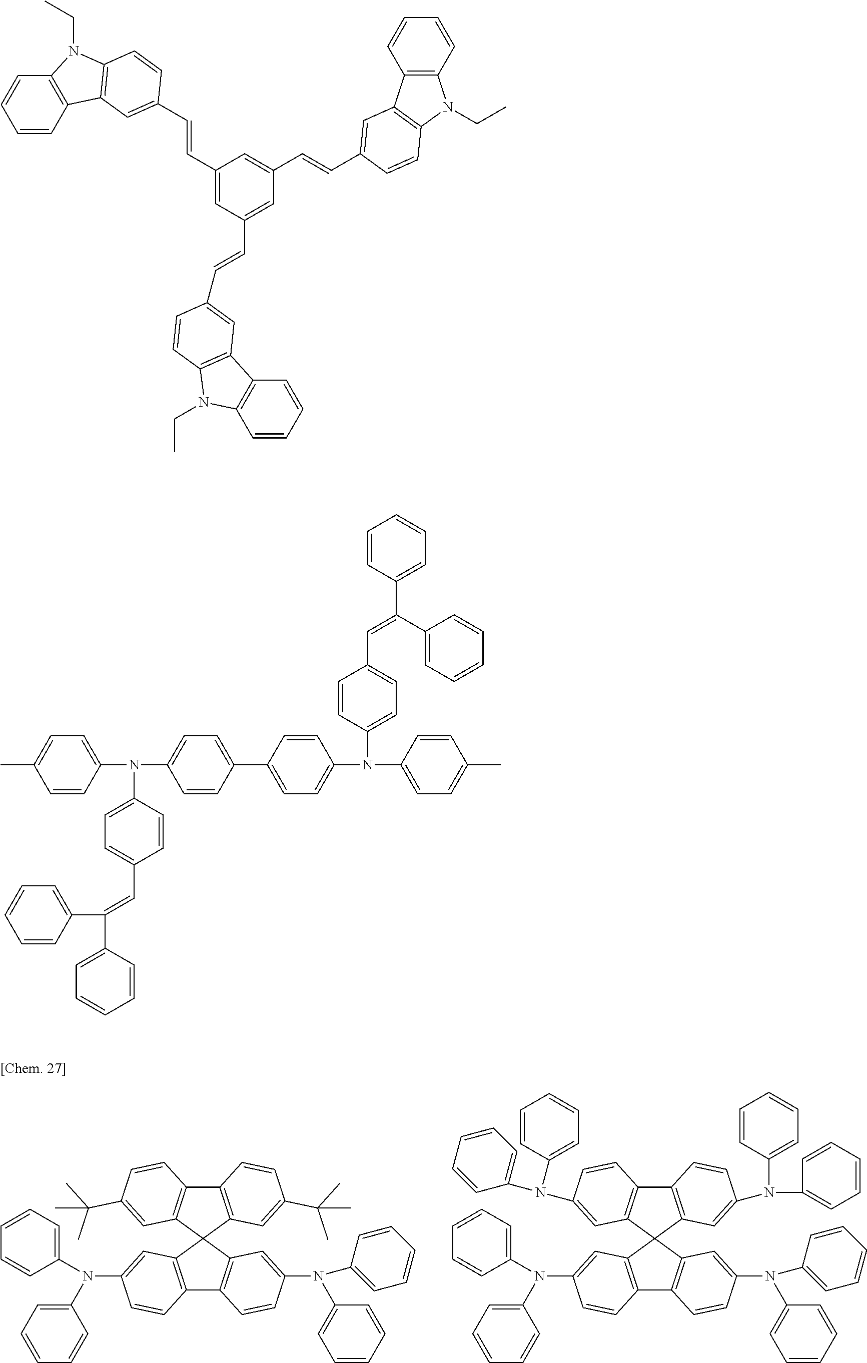

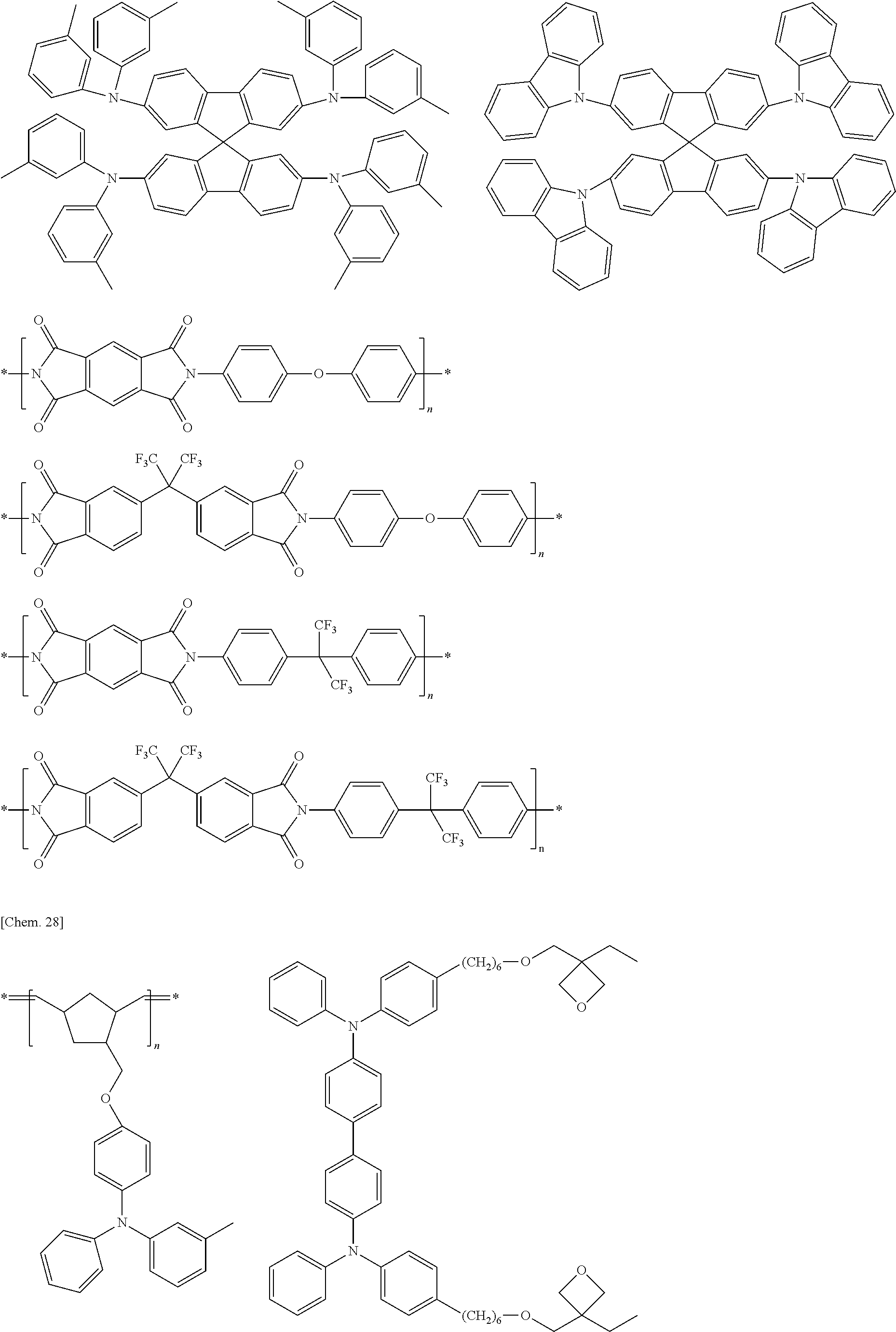

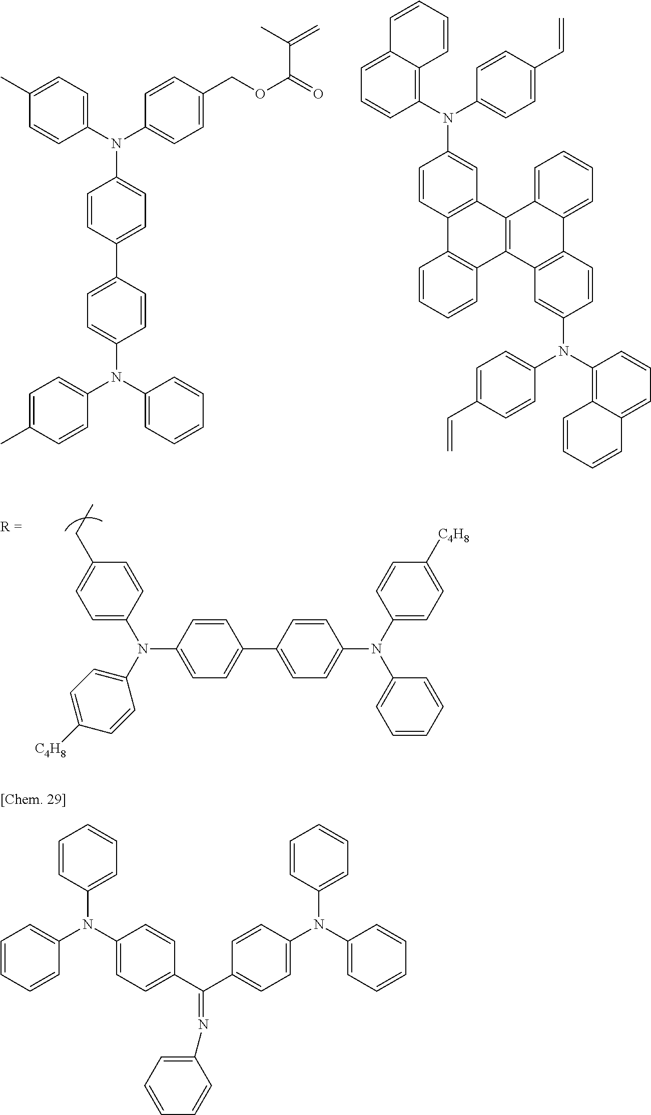

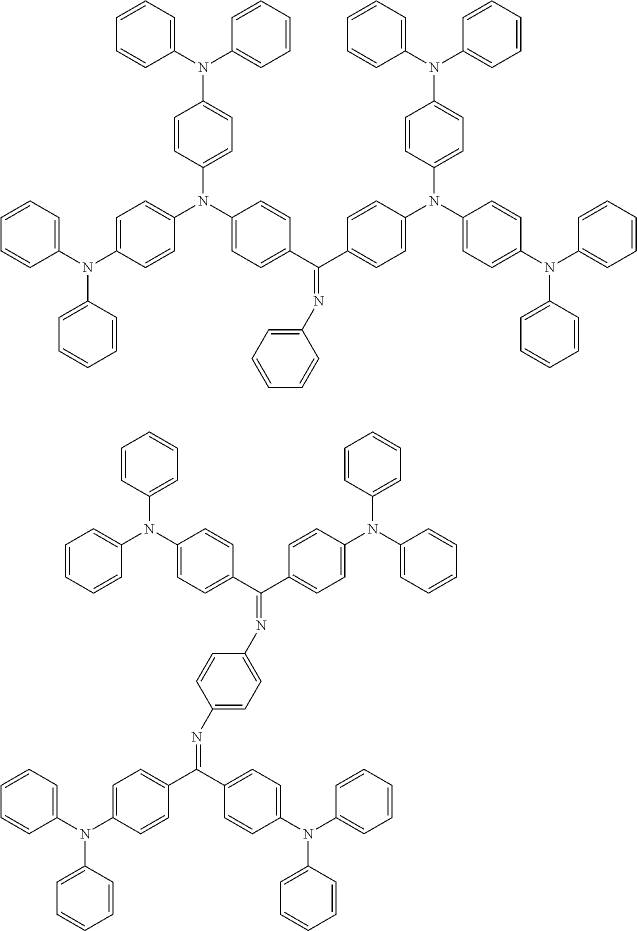 [SMILES: c1ccc(N=C(c2ccc(N(c3ccc(N(c4ccccc4)c4ccccc4)cc3)c3ccc(N(c4ccccc4)c4ccccc4)cc3)cc2)c2ccc(N(c3ccc(N(c4ccccc4)c4ccccc4)cc3)c3ccc(N(c4ccccc4)c4ccccc4)cc3)cc2)cc1 ; c1ccc(N(c2ccccc2)c2ccc(C(=Nc3ccc(N=C(c4ccc(N(c5ccccc5)c5ccccc5)cc4)c4ccc(N(c5ccccc5)c5ccccc5)cc4)cc3)c3ccc(N(c4ccccc4)c4ccccc4)cc3)cc2)cc1]

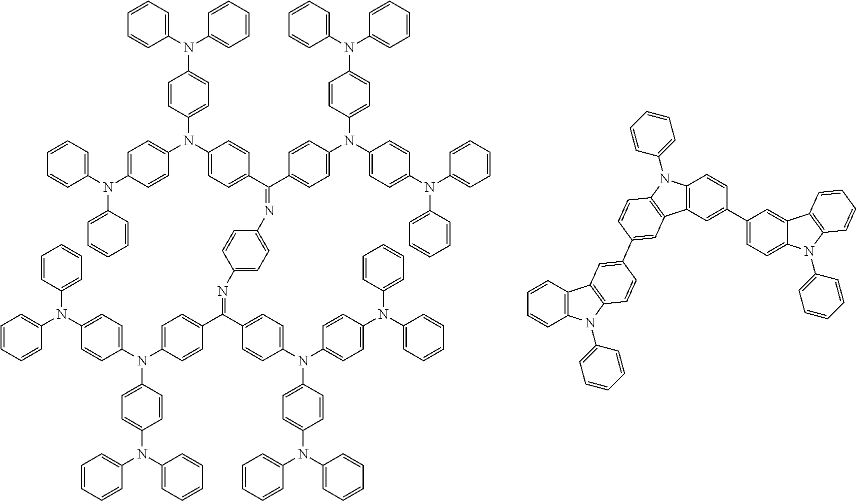 [SMILES: c1ccc(N(c2ccccc2)c2ccc(N(c3ccc(C(=Nc4ccc(N=C(c5ccc(N(c6ccc(N(c7ccccc7)c7ccccc7)cc6)c6ccc(N(c7ccccc7)c7ccccc7)cc6)cc5)c5ccc(N(c6ccc(N(c7ccccc7)c7ccccc7)cc6)c6ccc(N(c7ccccc7)c7ccccc7)cc6)cc5)cc4)c4ccc(N(c5ccc(N(c6ccccc6)c6ccccc6)cc5)c5ccc(N(c6ccccc6)c6ccccc6)cc5)cc4)cc3)c3ccc(N(c4ccccc4)c4ccccc4)cc3)cc2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

Preferred examples of a compound that may be used as the electron barrier material are shown below.

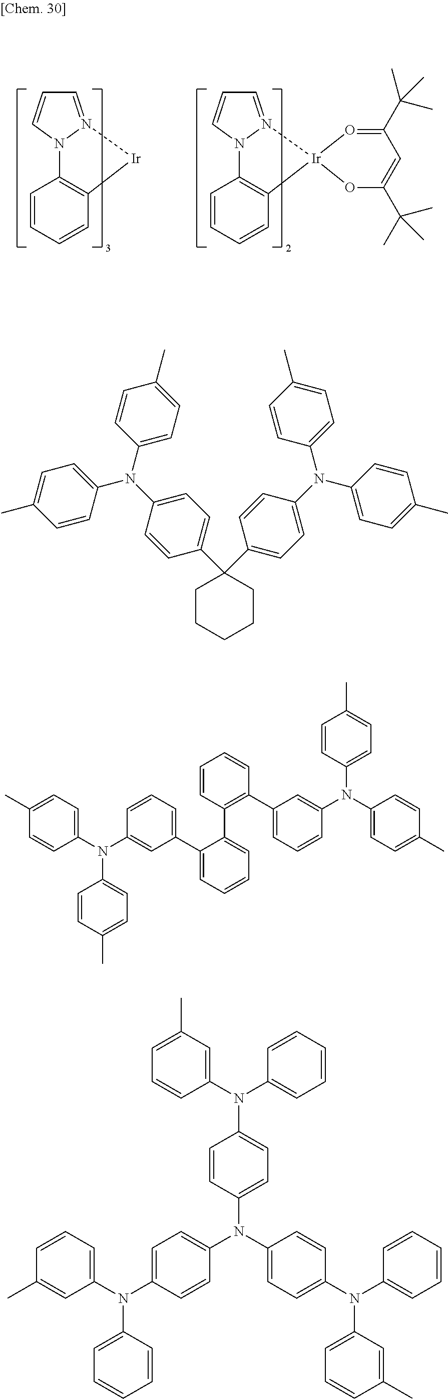

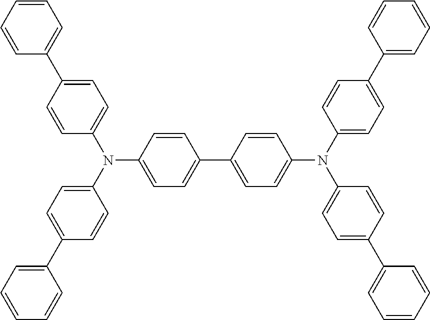 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

Preferred examples of a compound that may be used as the hole barrier material are shown below.

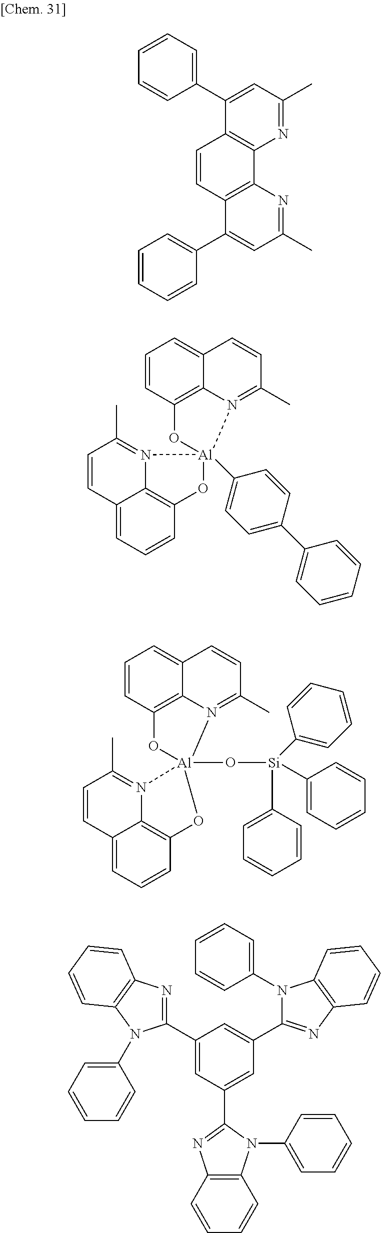

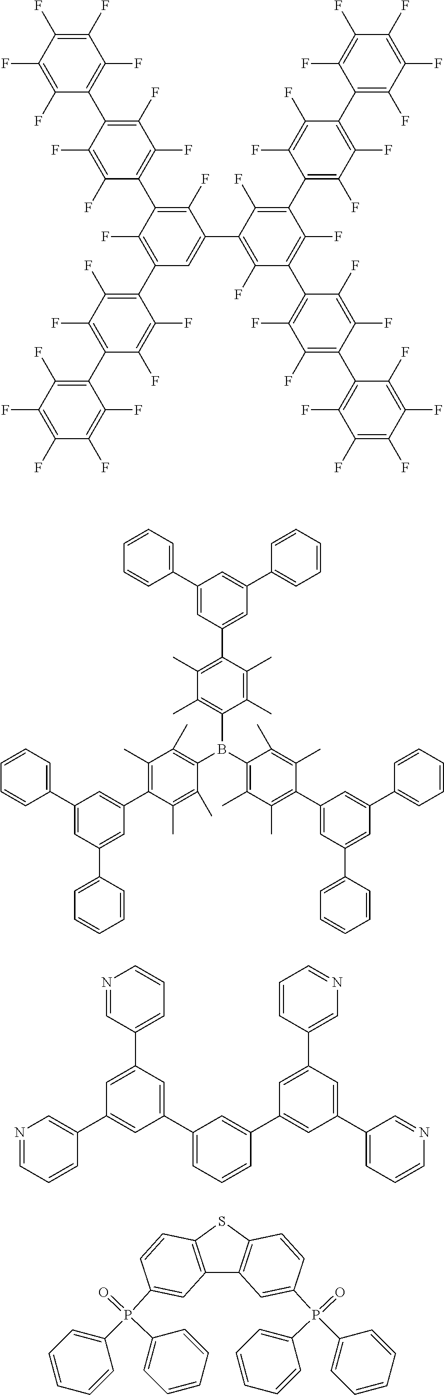

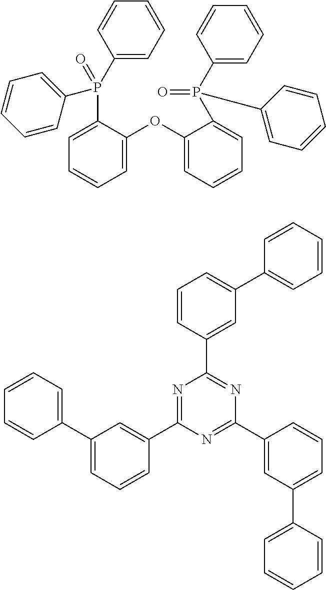 [SMILES: O=P(c1ccccc1)(c1ccccc1)c1ccccc1Oc1ccccc1P(=O)(c1ccccc1)c1ccccc1 ; c1ccc(-c2cccc(-c3nc(-c4cccc(-c5ccccc5)c4)nc(-c4cccc(-c5ccccc5)c4)n3)c2)cc1]

Preferred examples of a compound that may be used as the electron transporting material are shown below.

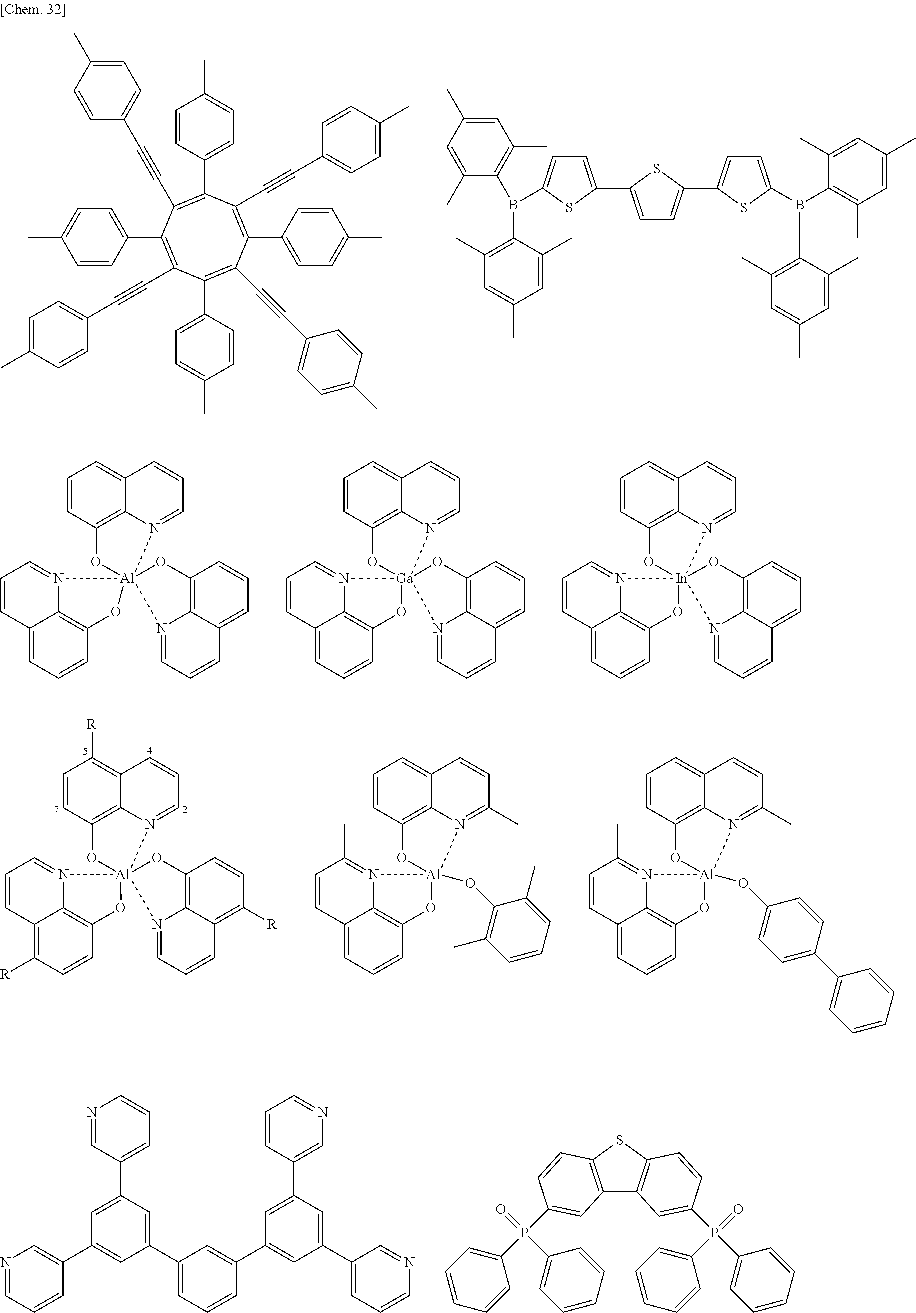

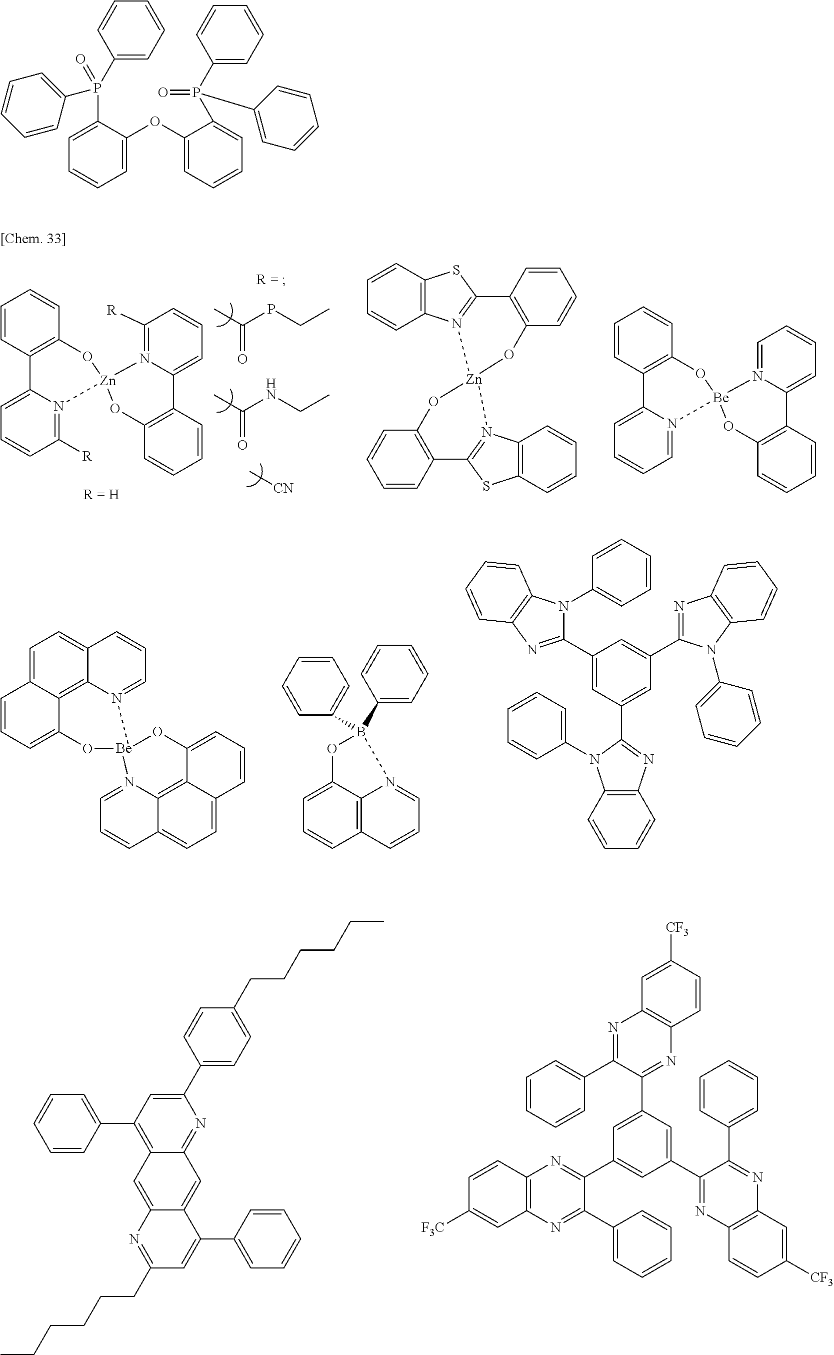

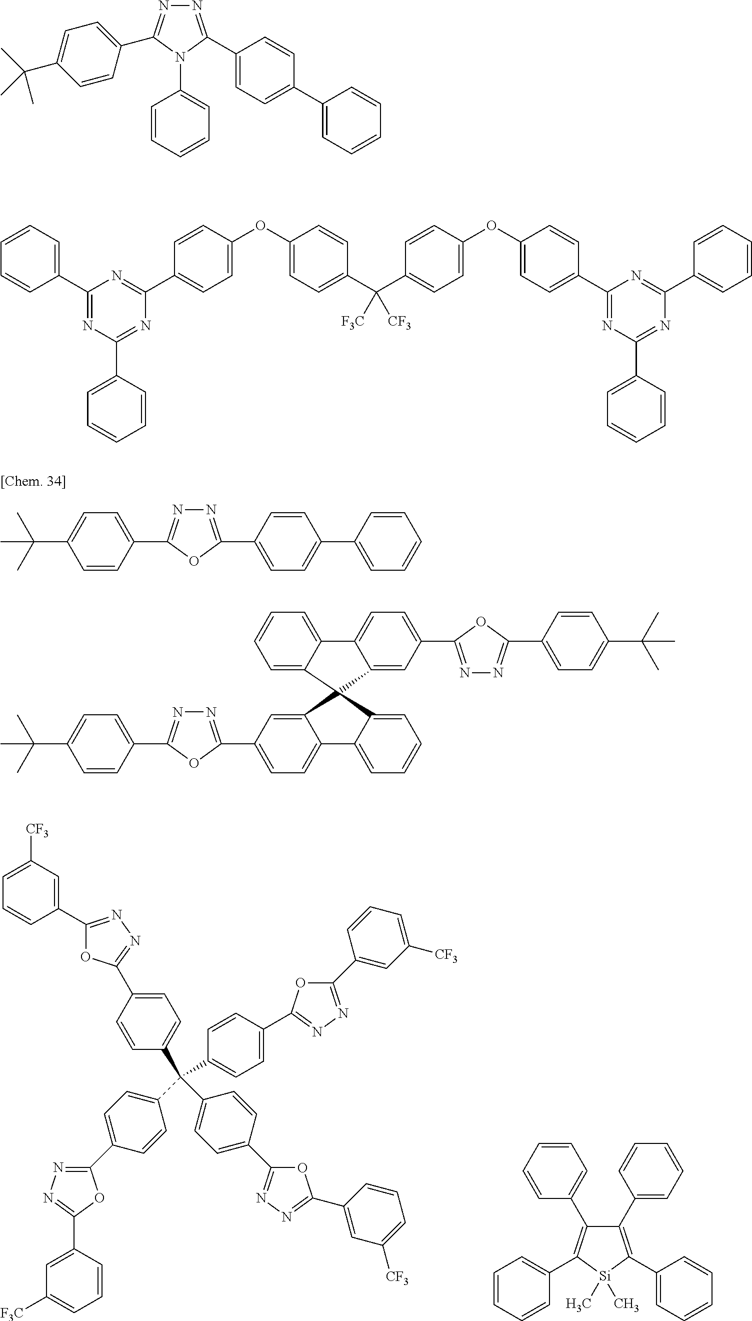

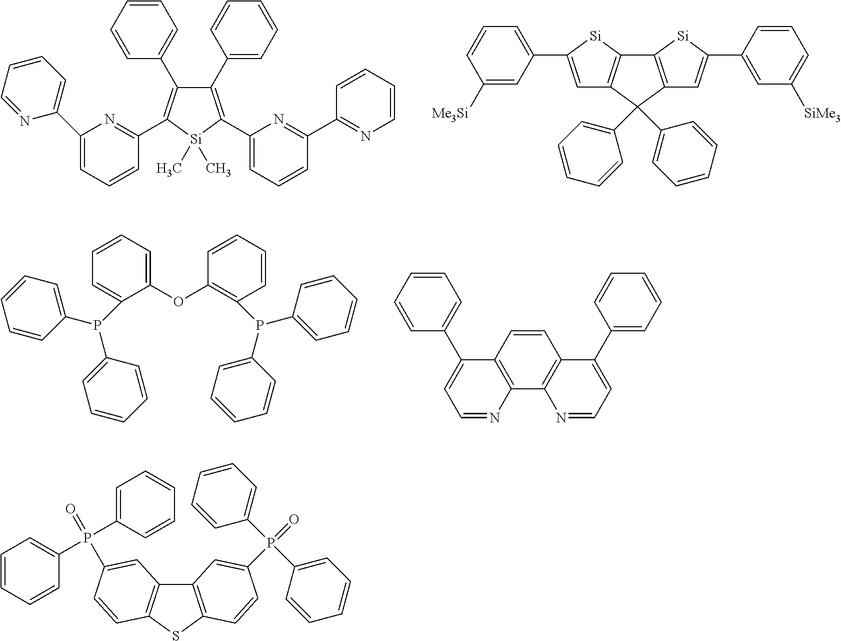

Preferred examples of a compound that may be used as the electron injection material are shown below.

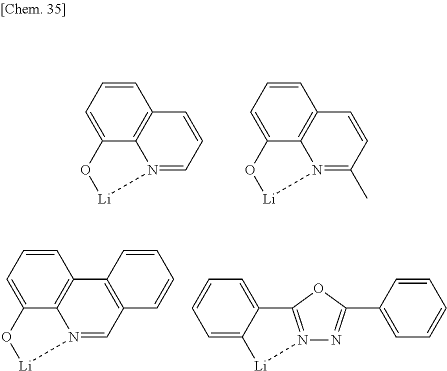

Preferred examples of a compound as a material that may be added are shown below. For example, the compound may be added as a stabilizing material.

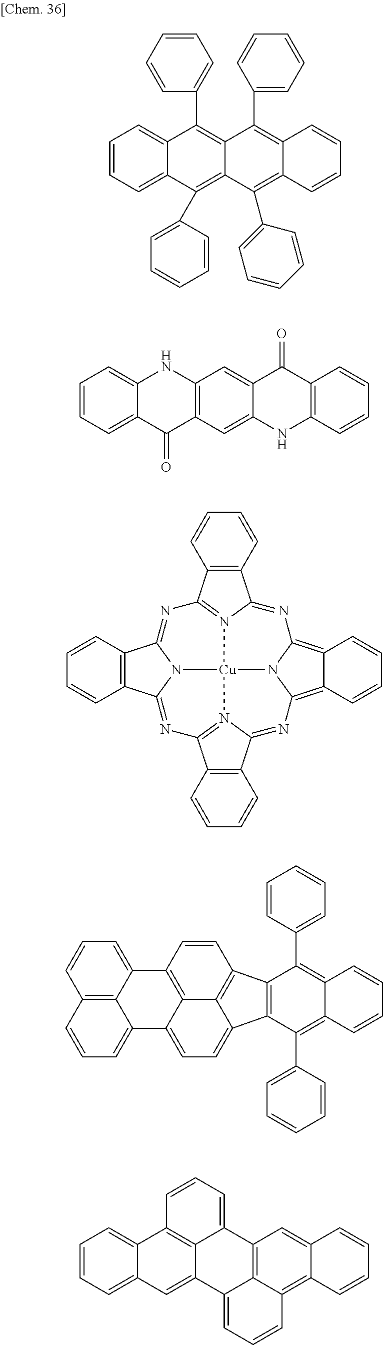

The organic electroluminescent device thus produced by the aforementioned method emits light on application of an electric field between the anode and the cathode of the device. In this case, when the light emission is caused by the excited single energy, light having a wavelength that corresponds to the energy level thereof may be confirmed as fluorescent light and delayed fluorescent light. When the light emission is caused by the excited triplet energy, light having a wavelength that corresponds to the energy level thereof may be confirmed as phosphorescent light. The normal fluorescent light has a shorter light emission lifetime than the delayed fluorescent light, and thus the light emission lifetime may be distinguished between the fluorescent light and the delayed fluorescent light.

The phosphorescent light may substantially not observed with a normal organic compound, such as the compound of the invention, at room temperature since the excited triplet energy is converted to heat of the like due to the instability thereof, and is immediately deactivated with a short lifetime. The excited triplet energy of the normal organic compound may be measured by observing light emission under an extremely low temperature condition.

The organic electroluminescent device of the invention may be applied to any of a single device, a device having a structure with plural devices disposed in an array, and a device having anodes and cathodes disposed in an X—Y matrix. According to the invention, an organic light emitting device that is largely improved in light emission efficiency may be obtained by adding the compound represented by the general formula (1) in the light emitting layer. The organic light emitting device, such as the organic electroluminescent device, of the invention may be applied to a further wide range of purposes. For example, an organic electroluminescent display apparatus may be produced with the organic electroluminescent device of the invention, and for the details thereof, reference may be made to S. Tokito, C. Adachi and H. Murata, “Yuki EL Display” (Organic EL Display) (Ohmsha, Ltd.). In particular, the organic electroluminescent device of the invention may be applied to organic electroluminescent illumination and backlight which are highly demanded.

### EXAMPLE

The features of the invention will be described more specifically with reference to synthesis examples and working examples below. The materials, processes, procedures and the like shown below may be appropriately modified unless they deviate from the substance of the invention. Accordingly, the scope of the invention is not construed as being limited to the specific examples shown below.

### Synthesis Example 1

### Synthesis of Compound 1

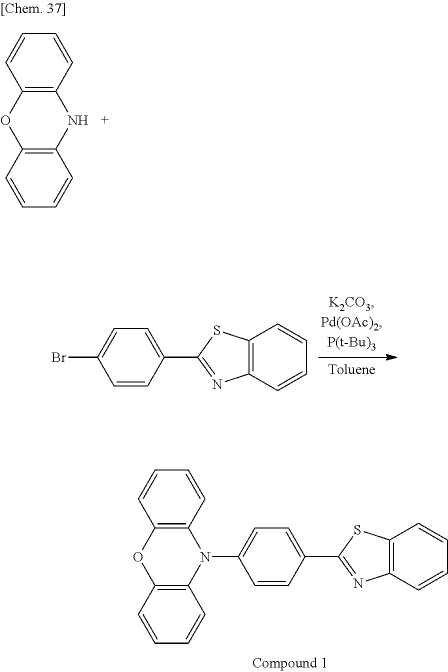 [SMILES: c1ccc2c(c1)Nc1ccccc1O2 ; Brc1ccc(-c2nc3ccccc3s2)cc1 ; c1ccc2c(c1)Oc1ccccc1N2c1ccc(-c2nc3ccccc3s2)cc1]

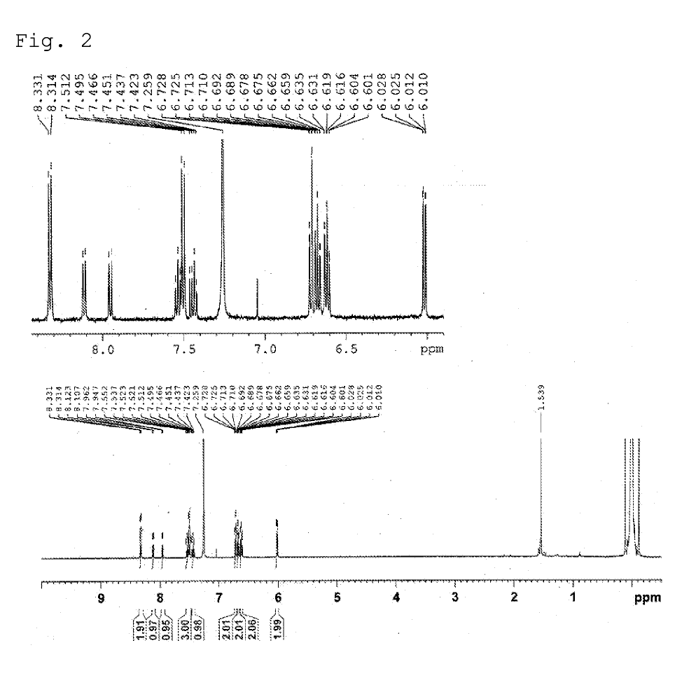

To a 100 ml two-neck flask having been substituted with nitrogen, phenoxazine (3.8 mmol, 0.70 g), 2-(4-bromophenyl)benzothiazol (2.5 mmol, 0.74 g) were poured. To the mixture, 10 mL of dry dehydrated toluene, potassium carbonate (7.2 mmol, 1.0 g), palladium acetate (0.25 mmol, 0.060 g), tri-tert-butylphosphine (0.25 mmol, 0.051 g) were added. The mixture was stirred in a nitrogen atmosphere at 100° C. for 15 hours. After stirring the mixture, 200 ml of ethyl acetate and saturated saline were added to the mixture, and an organic layer and a water layer were separated. The organic layer was dehydrated over magnesium sulfate. After the dehydration, and the mixture was filtered off under suction to provide a filtrate. The obtained filtrate was dissolved in chloroform and refined by silica gel column chromatography (developing solvent: chloroform/hexane=1/3 (v/v)). After the refining, the obtained fraction was condensed to collect a solid matter, and a yellow powder-like solid matter was obtained (yield amount: 0.78 g, yield: 78%). FIG. 2 shows ¹H-NMR (CDCl₃, 500 MHz).

### Synthesis Example 2

### Synthesis of Compound 2

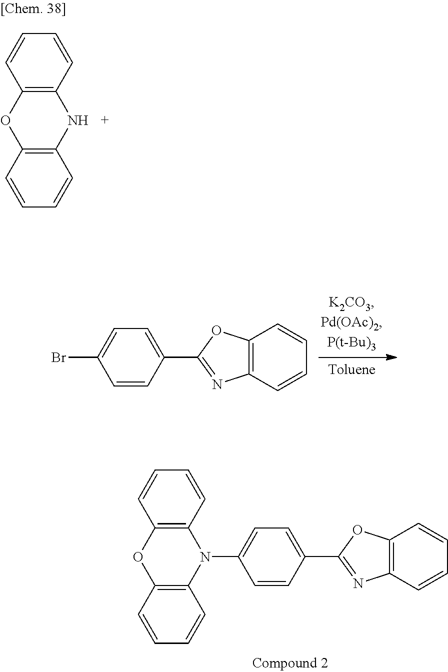 [SMILES: c1ccc2c(c1)Nc1ccccc1O2 ; Brc1ccc(-c2nc3ccccc3o2)cc1 ; c1ccc2c(c1)Oc1ccccc1N2c1ccc(-c2nc3ccccc3o2)cc1]

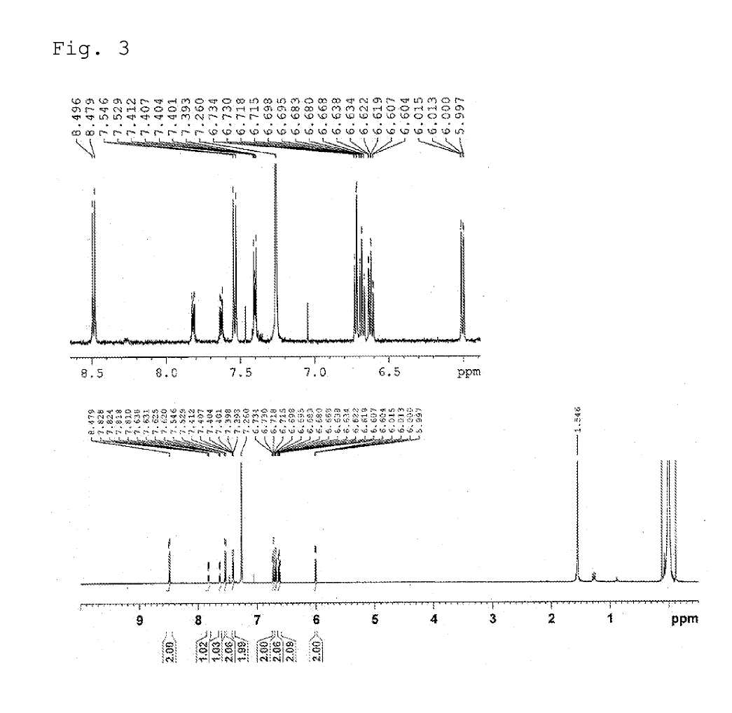

To a 100 ml two-neck flask having been substituted with nitrogen, phenoxazine (2.7 mmol, 0.50 g), 2-(4-bromophenyl)benzothiazol (2.7 mmol, 0.73 g) were poured. To the mixture, 10 mL of dry dehydrated toluene, potassium carbonate (8.0 mmol, 1.1 g), palladium acetate (0.85 mmol, 0.18 g), tri-tert-butylphosphine (1.0 mmol, 0.22 g) were added. The mixture was stirred in a nitrogen atmosphere at 100° C. for fifteen hours. After stirring the mixture, 200 ml of ethyl acetate and saturated saline were added to the mixture, and an organic layer and a water layer were separated. The organic layer was dehydrated over magnesium sulfate. After the dehydration, and the mixture was filtered off under suction to provide a filtrate. The obtained filtrate was dissolved in chloroform and then refined by silica gel column chromatography (developing solvent: chloroform/hexane=1/1 (v/v)). After the refining, the obtained fraction was condensed to collect a solid matter, and a yellow powder-like solid matter was obtained (yield amount: 0.65 g, yield: 65%). FIG. 3 shows ¹H-NMR (CDCl₃, 500 MHz).

### Example 1

### Production and Evaluation of Solution

A toluene solution of the compound 1 synthesized in Synthesis Example 1 (concentration: 10⁻⁵ mol/L) was prepared and irradiated with ultraviolet light at 300 K under bubbling with nitrogen, and thus fluorescent light having a peak wavelength of 512 nm was observed as shown in FIG. 4. The solution was observed with a compact fluorescence lifetime spectrometer (Quantaurus-tau, produced by Hamamatsu Photonics K.K.) before and after bubbling with nitrogen, thereby providing the transient decay curve shown in FIG. 5. The transient decay curve shows a result of measuring light emission lifetime, namely a process of deactivation of emission intensity, by irradiating the compound with excitation light. The light intensity attenuates in a monoexponential function manner in the case of an ordinary light emission of a single component (fluorescent light or phosphorescent light). This means that the light intensity linearly attenuates in the case in which the vertical axis of the graph is plotted in a semi-log manner. According to the transient decay curve of the compound 1 shown in FIG. 5, such a linear component (fluorescent light) is observed in an initial stage of the observation, and a component which deviates from the linearity appears several microseconds later. This corresponds to light emission of a delayed component, and a signal to be added to the initial component corresponds to a moderate curve with a bottom extending toward the longer-time side. By measuring the light emission lifetime as described above, it was confirmed that the compound 1 was a light emitting body that contained, in addition to the fluorescent component, the delayed component. That is, a short lifetime component with an excitation lifetime of 0.013 μs and a long lifetime component with an excitation lifetime of 39 μs were observed in the toluene solution of the compound 1. The photoluminescent quantum efficiency of the compound 1 in the toluene solution was measured at 300 K with an absolute PL quantum yields measurement system (Quantaurus-QY, produced by Hamamatsu Photonics K.K.), and was 16.0% before bubbling with nitrogen and 33.4% after bubbling with nitrogen.

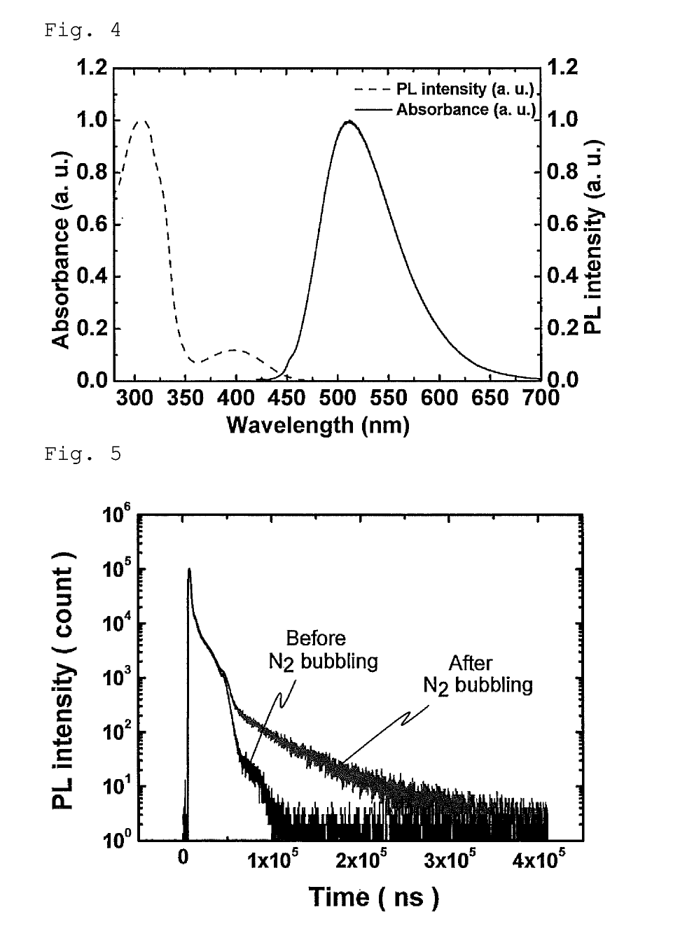

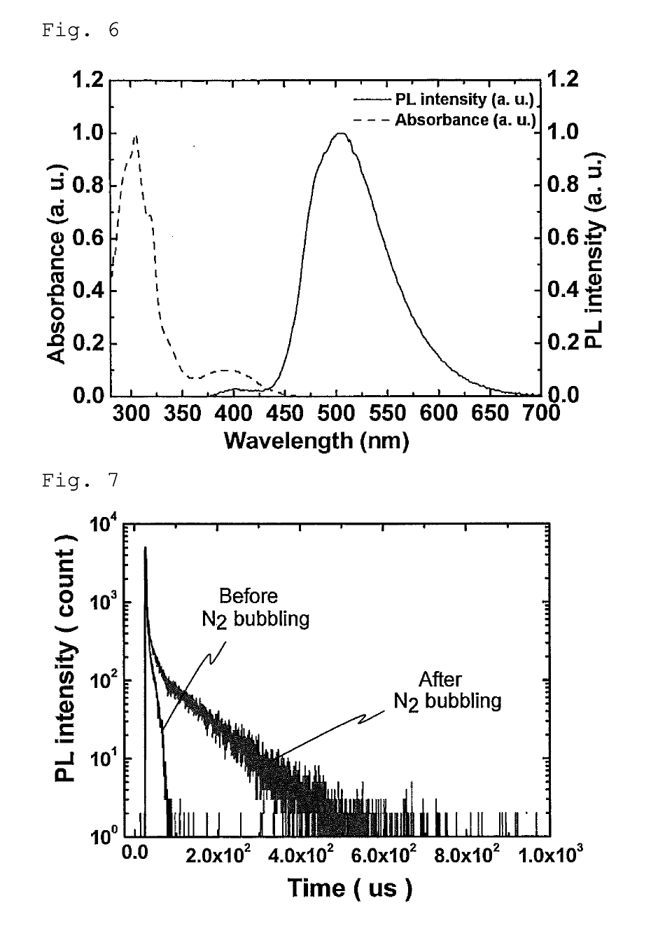

Similarly, a toluene solution was produced by using the compound 2 synthesized in Synthesis Example 2 instead of the compound 1 and was evaluated. FIG. 6 shows a light emission spectrum with a peak wavelength of 503 nm, and FIG. 7 shows a transient decay curve after bubbling with nitrogen. A short lifetime component with an excitation lifetime of 0.012 μs and a long lifetime component with an excitation lifetime of 140 μs were observed. The photoluminescence quantum efficiency was 17.5% before bubbling with nitrogen and 24.7% after bubbling with nitrogen.

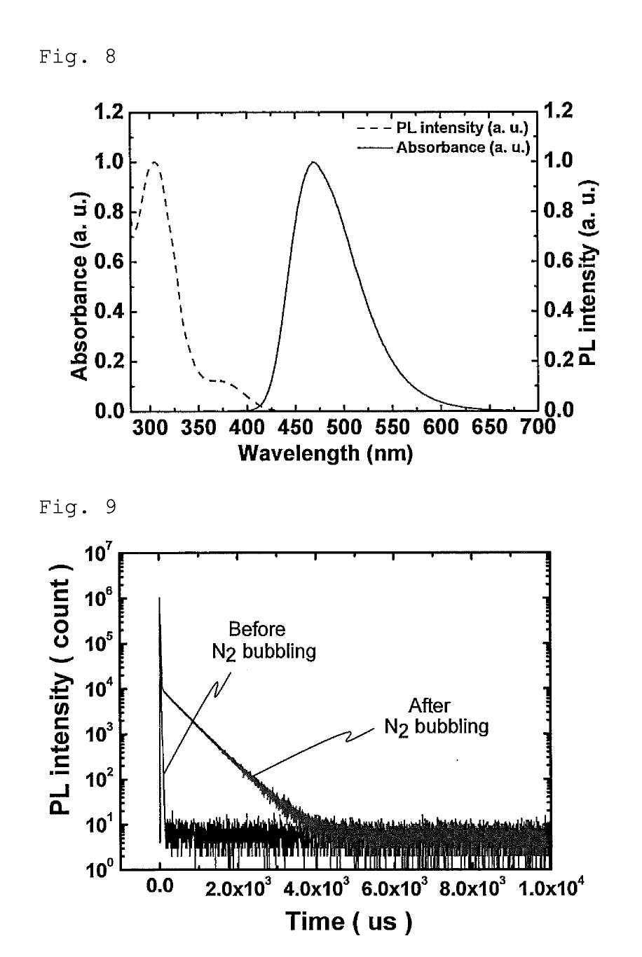

Similarly, a toluene solution was produced by using the compound 3 and was evaluated. FIG. 8 shows a light emission spectrum with a peak wavelength of 468 nm, and FIG. 9 shows a transient decay curve after bubbling with nitrogen. A short lifetime component with an excitation lifetime of 0.01 μs and a long lifetime component with an excitation lifetime of 490 μs were observed. The photoluminescence quantum efficiency was 14.1% before bubbling with nitrogen and 21.1% after bubbling with nitrogen.

### Comparative Example 1

### Production and Evaluation of Solution

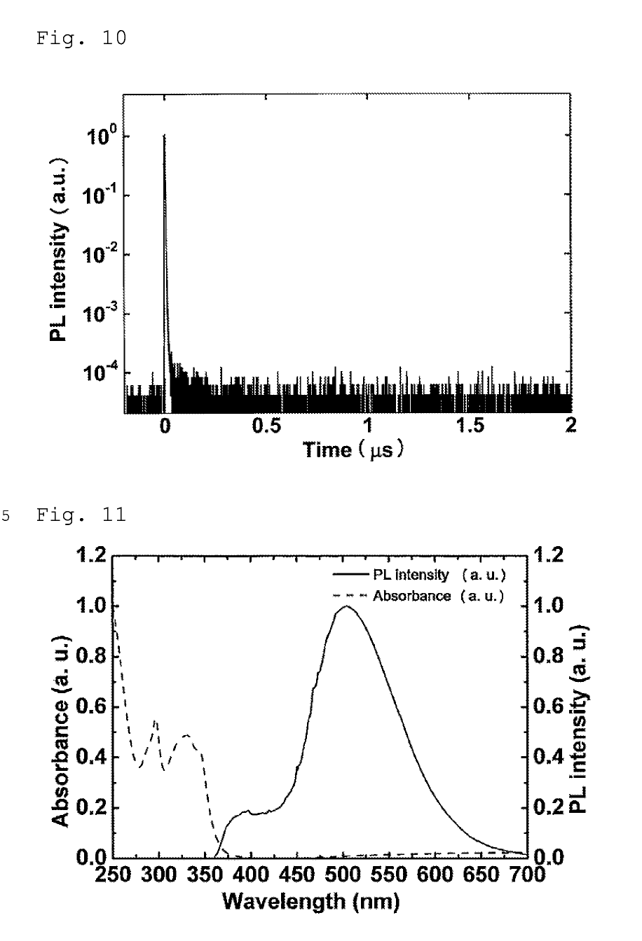

A toluene solution of a comparative compound that has the following structure was produced in the same manner as in Example 1. Transient decay curves before and after bubbling with nitrogen overlap with each other as shown in FIG. 10. Since no clear delayed component was observed after bubbling with nitrogen, it was confirmed that the comparative compound was not a delayed fluorescent body.

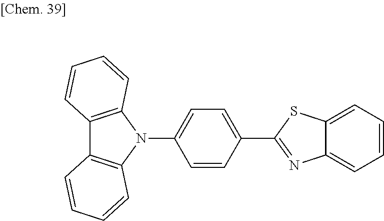 [SMILES: c1ccc2sc(-c3ccc(-n4c5ccccc5c5ccccc54)cc3)nc2c1]

### Example 2

### Production and Evaluation of Thin Film Organic Photoluminescent Device (Thin Film)

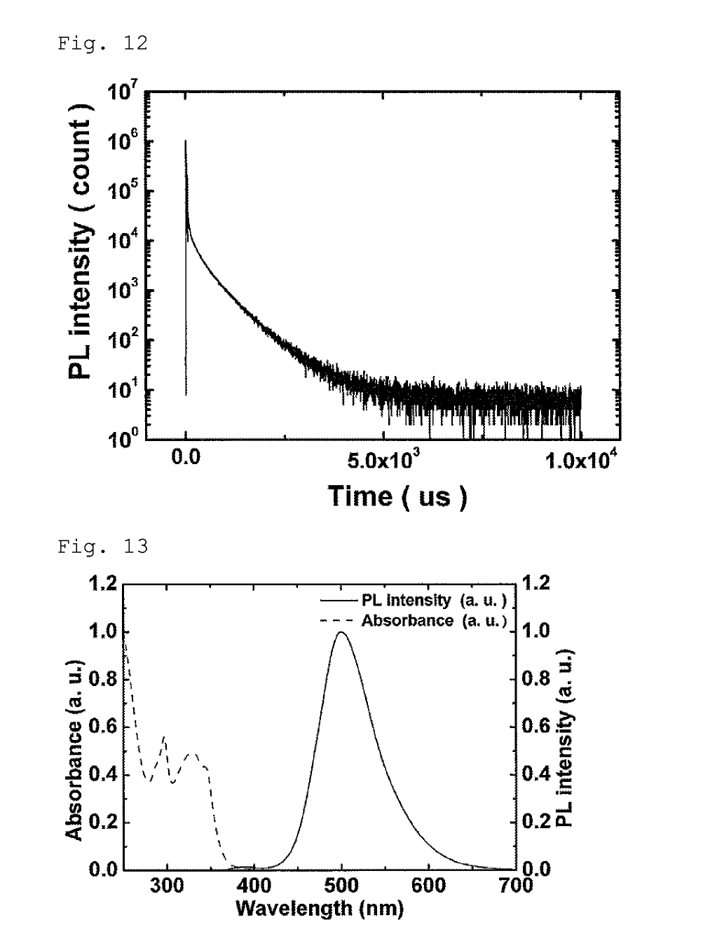

On a silicon substrate, the compound 1 and CBP were vapor-deposited from separate vapor deposition sources respectively by a vacuum vapor deposition method under condition of a vacuum degree of 5.0×10⁻⁴ Pa, thereby forming a thin film having a thickness of 100 nm and a concentration of the compound 1 of 6.0% by weight at a rate of 0.3 nm/sec, which was designated as an organic photoluminescent device. Measurement was made by using the same measurement apparatus as that in Example 1, and a light emission spectrum with a peak wavelength of 504 nm was obtained (FIG. 11). The photoluminescent quantum efficiency at 300 K was 62.0%. The lifetime of excitrons was measured by using the same measurement apparatus as that in Example 1, and the transient decay curve shown in FIG. 12 was obtained. A short lifetime component had an excitation lifetime of 0.013 μs, and a long lifetime component had an excitation lifetime of 576 μs.

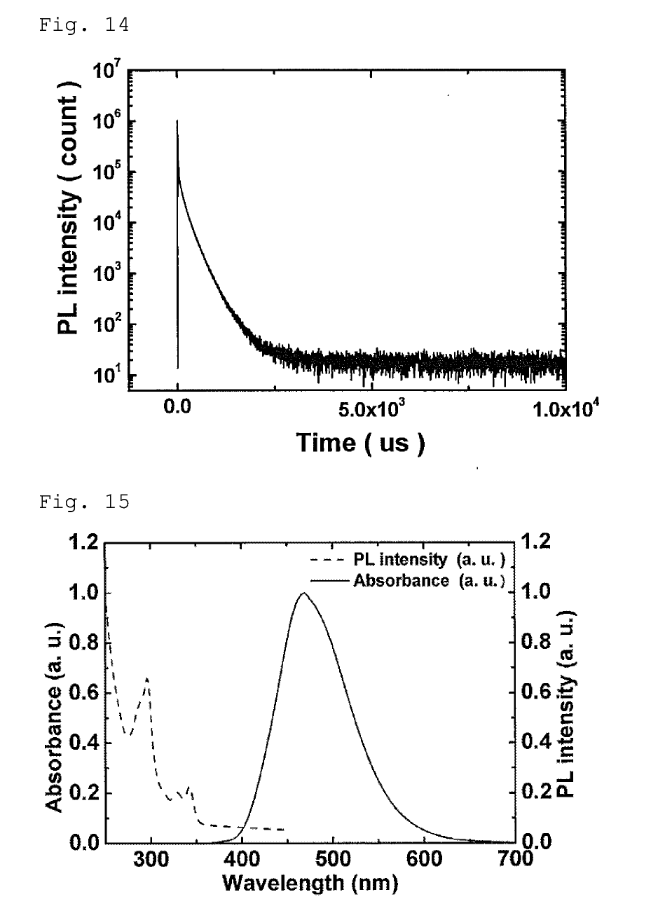

Similarly, a thin film was formed by using the compound 2 and evaluated, a light emission spectrum with a peak wavelength of 498 nm was obtained (FIG. 13), and a transient decay curve shown in FIG. 14 was obtained. The photoluminescence quantum efficiency at 300 K was 65.0%. A short lifetime component had an excitation lifetime of 0.013 μs, and a long lifetime component had an excitation lifetime of 300 μs.

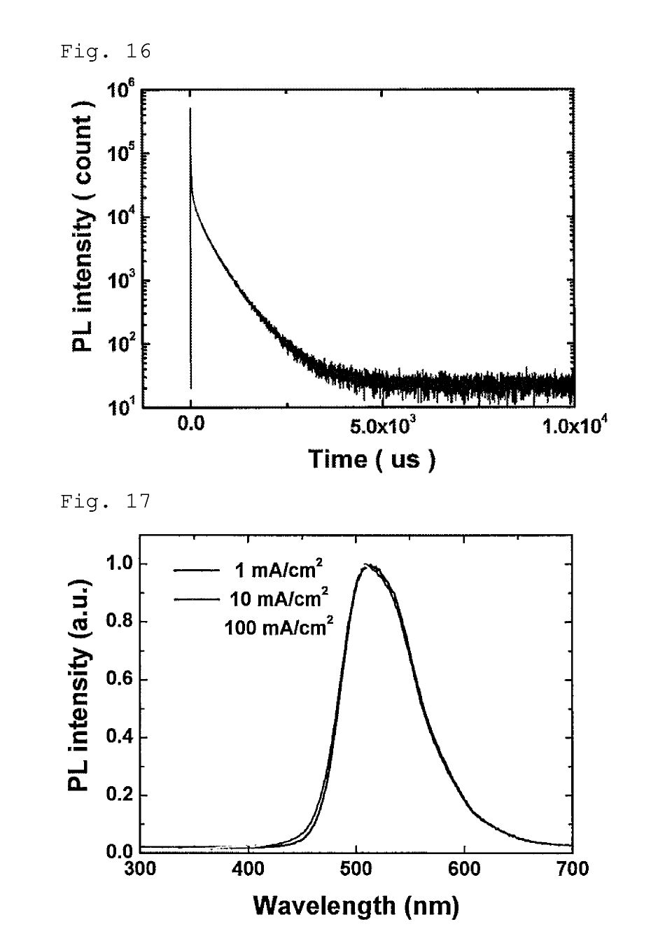

Similarly, a thin film was formed by using the compound and evaluated, a light emission spectrum with a peak wavelength of 469 nm was obtained (FIG. 15), and a transient decay curve shown in FIG. 16 was obtained. The photoluminescence quantum efficiency at 300 K was 35%. A short lifetime component had an excitation lifetime of 0.013 μs, and along lifetime component had an excitation lifetime of 462 μs.

### Example 3

### Production and Evaluation of Organic Electroluminescent Device

Thin films each were formed by a vacuum vapor deposition method at a vacuum degree of 5.0×10⁻⁴ Pa on a glass substrate having formed thereon an anode formed of indium tin oxide (ITO) having a thickness of 100 nm. First, α-NPD was formed to a thickness of 35 nm on ITO. The compound 1 and CBP were then vapor-deposited from separate vapor deposition sources respectively to form a layer having a thickness of 15 nm, which was designated as a light emitting layer. The concentration of the compound 1 herein was 6.0% by weight. TPBi was then formed to a thickness of 65 nm, lithium fluoride (LiF) was further vapor-deposited to a thickness of 0.8 nm, and then aluminum (Al) was vapor-deposited to a thickness of 80 nm, which was designated as a cathode, thereby completing an organic electroluminescent device.

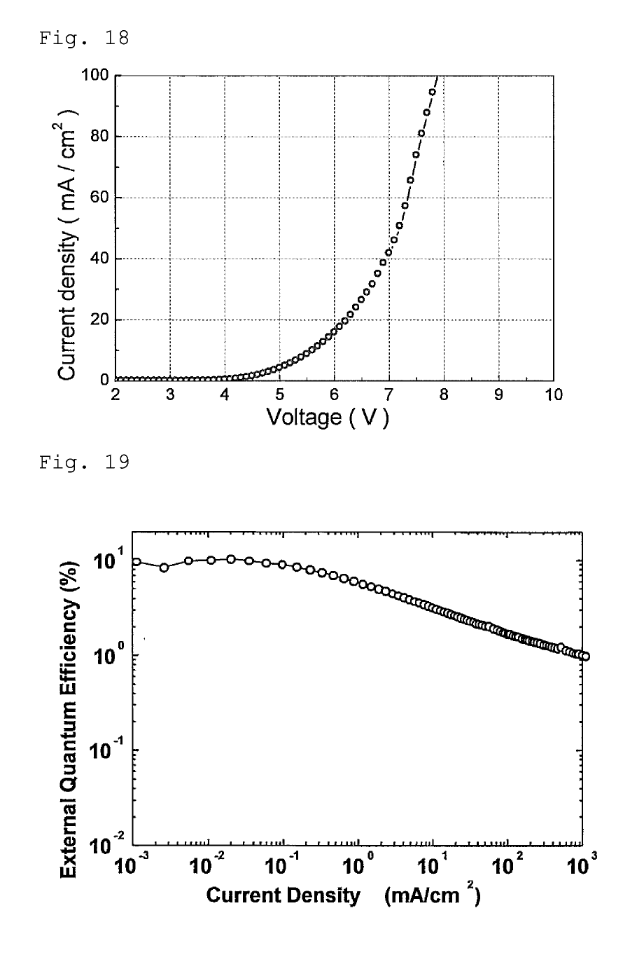

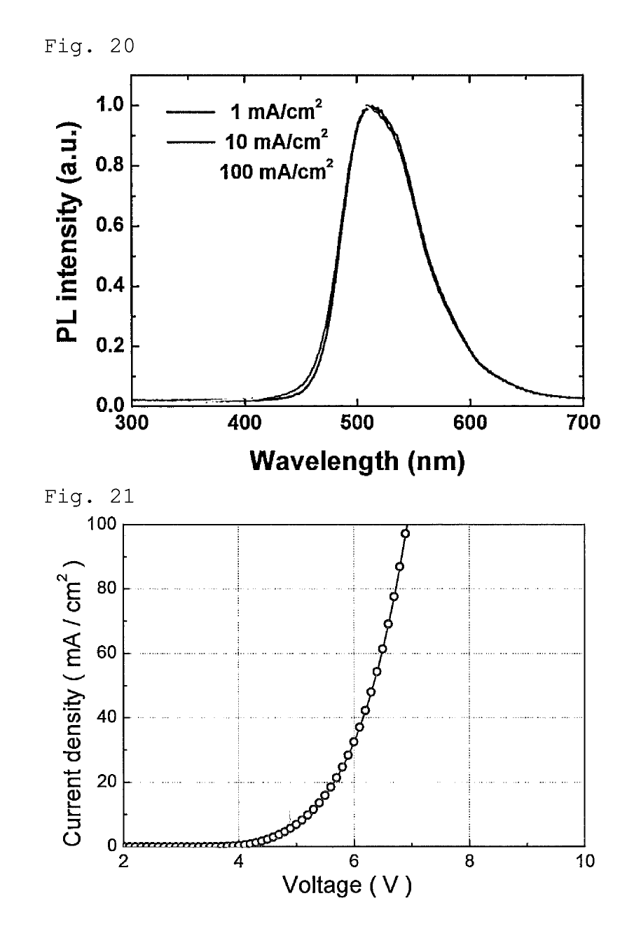

The organic electroluminescent device thus produced was measured with Semiconductor Parameter Analyzer (E5273A, produced by Agilent Technologies, Inc.), Optical Power Meter (1930C, produced by Newport Corporation) and Fiber Optic Spectrometer (USB2000, produced by Ocean Optics, Inc.), and thus light emission with a peak wavelength of 508 nm was observed as shown in FIG. 17. FIG. 18 shows the electric current density-voltage characteristics, and FIG. 19 shows the electric current density-external quantum efficiency characteristics. The organic electroluminescent device using the compound 1 as a light emitting material achieved a high external quantum efficiency of 10.29%. On the assumption that a balanced ideal organic electroluminescence device is produced as a trial by using a fluorescent material with light emission quantum efficiency of 100%, and that light extraction efficiency is from 20% to 30%, then the external quantum efficiency of the fluorescent light emission becomes from 5% to 7.5%. This value is generally considered to be a theoretical limitation value of the external quantum efficiency of the organic electroluminescence device using the fluorescent material. The organic electroluminescence element of the invention, which uses the compound 1, is excellent in high external quantum efficiency exceeding the theoretical limitation value.

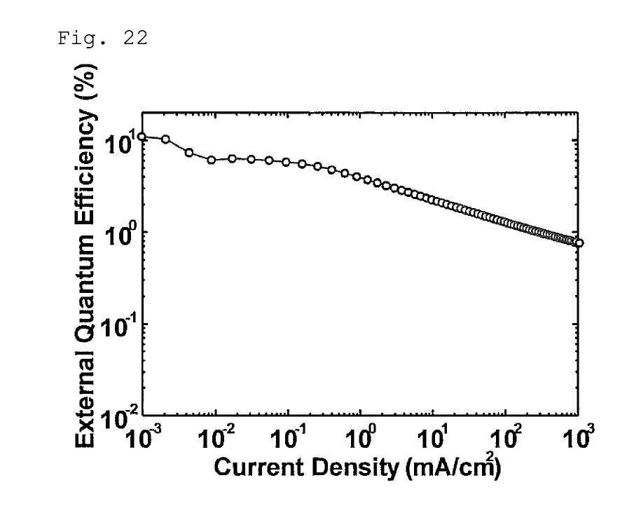

Similarly, an organic electroluminescent device was further produced by using the compound 2 and evaluated, and light emission with a peak wavelength of 504 nm was observed as shown in FIG. 20. FIG. 21 shows the electric current density-voltage characteristics, and FIG. 22 shows the electric current density-external quantum efficiency characteristics. The organic electroluminescent device using the compound 2 as a light emitting material achieved a high external quantum efficiency of 6.31%.

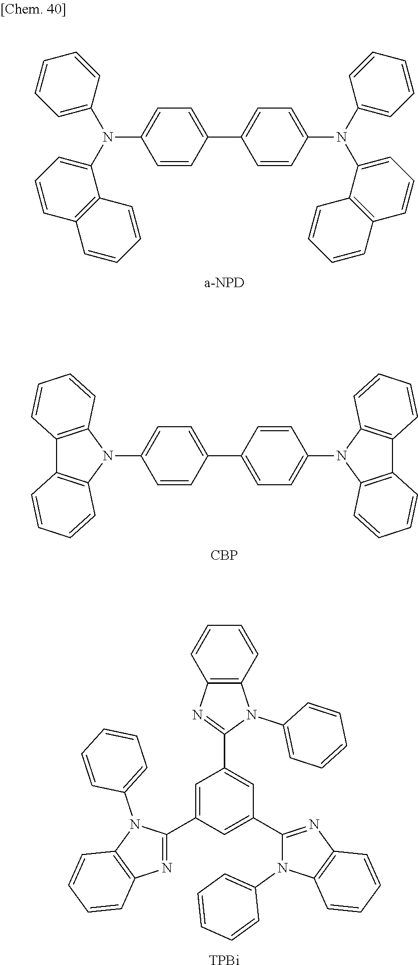

### INDUSTRIAL APPLICABILITY

The compound of represented by the general formula (1) is useful as a light emitting material. Accordingly, the compound represented by the general formula (1) may be effectively used as a light emitting material of an organic light emitting device, such as an organic electroluminescent device. The compound represented by the general formula (1) includes a compound that emits delayed fluorescent light, and an organic light emitting device having a high light emission efficiency may be provided. Accordingly, the invention has high industrial applicability.

### REFERENCE SIGNS LIST

1 substrate
2 anode
3 hole injection layer
4 hole transporting layer
5 light emitting layer
6 electron transporting layer
7 cathode

1. A light emitting material comprising a compound represented by the following general formula (1): 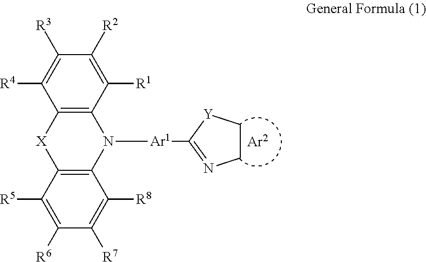
wherein in the general formula (1), X represents O, S, N—R¹¹, C═O, C(R¹²)(R¹³), or Si(R¹⁴)(R¹⁵);
Y represents O, S, or N—R¹⁶; Ar¹ represents a substituted or unsubstituted arylene group; Ar² represents an aromatic ring or a heteroaromatic ring; provided that when X is O or S, then R¹⁶ is not a phenyl group; and R¹ to R⁸, R¹¹ and R¹⁴ to R¹⁶ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted acyl group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkoxycarbonyl group, a substituted or unsubstituted alkylsulfonyl group, a substituted or unsubstituted haloalkyl group, a substituted or unsubstituted amide group, a substituted or unsubstituted alkylamide group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, R¹² and R¹³ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkylsulfonyl group, a substituted or unsubstituted haloalkyl group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, provided that R¹ and R², R² and R³, R³ and R⁴, R⁵ and R⁶, R⁶ and R⁷, R⁷ and R⁸ each may be bonded to each other to form a cyclic structure.

2. The light emitting material according to claim 1, wherein the compound represented by the general formula (1) is a compound represented by the following general formula (2): 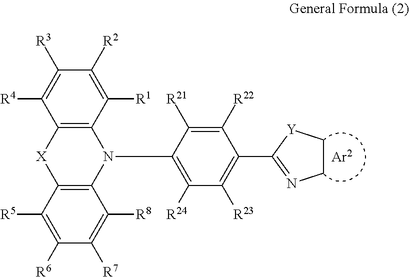
wherein in the general formula (2), X represents O, S, N—R¹¹, C═O, C(R¹²)(R¹³), or Si(R¹⁴)(R¹⁵);
Y represents O, S, or N—R¹⁶; provided that when X is O or S, then R¹⁶ is not a phenyl group; Ar² represents an aromatic ring or a heteroaromatic ring; and R¹ to R⁸, R¹¹, R¹⁴ to R¹⁶, and R²¹ to R²⁴ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted acyl group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkoxycarbonyl group, a substituted or unsubstituted alkylsulfonyl group, a substituted or unsubstituted haloalkyl group, a substituted or unsubstituted amide group, a substituted or unsubstituted alkylamide group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, R¹² and R¹³ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkylsulfonyl group, a substituted or unsubstituted haloalkyl group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, provided that R¹ and R², R² and R³, R³ and R⁴, R⁵ and R⁶, R⁶ and R⁷, R⁷ and R⁸, R²¹ and R²², and R²³ and R²⁴ each may be bonded to each other to form a cyclic structure.

3. The light emitting material according to claim 1, wherein the compound represented by the general formula (1) is a compound represented by the following general formula (3): 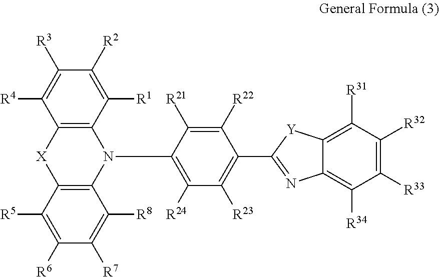
wherein in the general formula (3), X represents O, S, N—R¹¹, C═O, C(R¹²)(R¹³), or Si(R¹⁴)(R¹⁵);
Y represents O, S, or N—R¹⁶; provided that when X is O or S, then R¹⁶ is not a phenyl group; and
R¹ to R⁸, R¹¹, R¹⁴ to R¹⁶, R²¹ to R²⁴, and R³¹ to R³⁴ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted acyl group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkoxycarbonyl group, a substituted or unsubstituted alkylsulfonyl group, a substituted or unsubstituted haloalkyl group, a substituted or unsubstituted amide group, a substituted or unsubstituted alkylamide group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, R¹² and R¹³ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkylsulfonyl group, a substituted or unsubstituted haloalkyl group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, provided that R¹ and R², R² and R³, R³ and R⁴, R⁵ and R⁶, R⁶ and R⁷, R⁷ and R⁸, R²¹ and R²², R²³ and R²⁴, R³¹ and R³², R³² and R³³, and R³³ and R³⁴ each may be bonded to each other to form a cyclic structure.

4. The light emitting material according to claim 1, wherein X is O or S.

5. The light emitting material according to claim 1, wherein Y is O, S, or N—R¹⁶, and R¹⁶ represents a substituted or an unsubstituted aryl group.

6. The light emitting material according to claim 1, wherein R¹ to R⁸ each independently represent a hydrogen atom, a fluorine atom, a chlorine atom, a cyano group, a substituted or unsubstituted alkyl group having from 1 to 10 carbon atoms, a substituted or unsubstituted alkoxy group having from 1 to 10 carbon atoms, a substituted or unsubstituted dialkylamino group having from 1 to 10 carbon atoms, a substituted or unsubstituted diarylamino group having from 12 to 40 carbon atoms, a substituted or unsubstituted aryl group having from 6 to 15 carbon atoms, or a substituted or unsubstituted heteroaryl group having from 3 to 12 carbon atoms.

7. A delayed fluorescent emitter consisting of a compound represented by the following general formula (1): 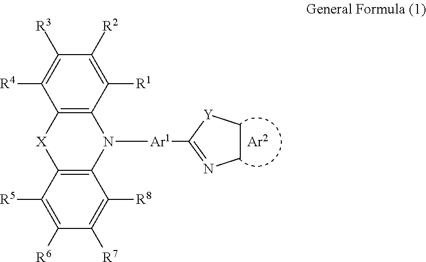
wherein in the general formula (1), X represents O, S, N—R¹¹, C═O, C(R¹²)(R¹³), or Si(R¹⁴)(R¹⁵);
Y represents O, S, or N—R¹⁶; Ar¹ represents a substituted or unsubstituted arylene group; Ar² represents an aromatic ring or a heteroaromatic ring; and R¹ to R⁸, and R¹¹ to R¹⁶ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted acyl group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkoxycarbonyl group, a substituted or unsubstituted alkylsulfonyl group, a substituted or unsubstituted haloalkyl group, a substituted or unsubstituted amide group, a substituted or unsubstituted alkylamide group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, R¹² and R¹³ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkylsulfonyl group, a substituted or unsubstituted haloalkyl group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, provided that R¹ and R², R² and R³, R³ and R⁴, R⁵ and R⁶, R⁶ and R⁷, R⁷ and R⁸ each may be bonded to each other to form a cyclic structure.

8. A compound represented by the following general formula (1′). 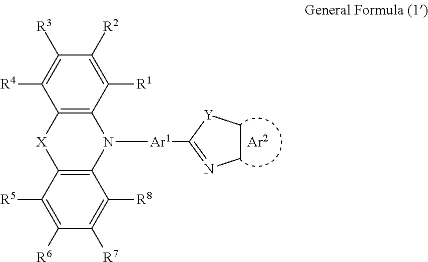
wherein in the general formula (1′), X represents O, S, N—R¹¹, C═O, C(R¹²)(R¹³), or Si(R¹⁴)(R¹⁵); Y represents O, S, or N—R¹⁶; Ar¹ represents a substituted or unsubstituted arylene group; Ar² represents an aromatic ring or a heteroaromatic ring; and R¹ to R⁸ and R¹¹ to R¹⁶ each independently represent a hydrogen atom or a substituent, provided that R¹⁶ is not a phenyl group when X is O or S, and Y is not S when X is C(R¹²)(R¹³), R¹ to R⁸, R¹¹ and R¹⁴ to R¹⁶ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted acyl group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkoxycarbonyl group, a substituted or unsubstituted alkylsulfonyl group, a substituted or unsubstituted haloalkyl group, a substituted or unsubstituted amide group, a substituted or unsubstituted alkylamide group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, R¹² and R¹³ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkylsulfonyl group, a substituted or unsubstituted haloalkyl group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, and R¹ and R², R² and R³, R³ and R⁴, R⁵ and R⁶, R⁶ and R⁷, R⁷ and R⁸ each may be bonded to each other to form a cyclic structure.

9. An organic light emitting device comprising a substrate having thereon a light emitting layer that comprises as a light emitting material
a compound represented by the following general formula (1): 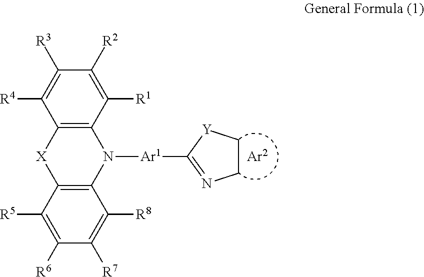
wherein in the general formula (1), X represents O, S, N—R¹¹, C═O, C(R¹²)(R¹³), or Si(R¹⁴)(R¹⁵);
Y represents O, S, or N—R¹⁶; Ar¹ represents a substituted or unsubstituted arylene group; Ar² represents an aromatic ring or a heteroaromatic ring; provided that when X is O or S, then R¹⁶ is not a phenyl group; and R¹ to R⁸, R¹¹ and R¹⁴ to R¹⁶ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted acyl group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkoxycarbonyl group, a substituted or unsubstituted alkylsulfonyl group, a substituted or unsubstituted haloalkyl group, a substituted or unsubstituted amide group, a substituted or unsubstituted alkylamide group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, R¹² and R¹³ each independently represent a hydrogen atom, a hydroxyl group, a halogen atom, a cyano group, a substituted or unsubstituted alkyl group, a substituted or unsubstituted alkoxy group, a substituted or unsubstituted alkylthio group, a substituted or unsubstituted alkyl-substituted amino group, a substituted or unsubstituted aryl-substituted amino group, a substituted or unsubstituted aryl group, a substituted or unsubstituted heteroaryl group, a substituted or unsubstituted carbazolyl group, a substituted or unsubstituted alkenyl group, a substituted or unsubstituted alkynyl group, a substituted or unsubstituted alkylsulfonyl group, a substituted or unsubstituted haloalkyl group, a substituted or unsubstituted trialkylsilyl group, a substituted or unsubstituted trialkylsilylalkyl group, a substituted or unsubstituted trialkylsilylalkenyl group, a substituted or unsubstituted trialkylsilylalkynyl group or a nitro group, provided that R¹ and R², R² and R³, R³ and R⁴, R⁵ and R⁶, R⁶ and R⁷, R⁷ and R⁸ each may be bonded to each other to form a cyclic structure.

10. The organic light emitting device according to claim 9, wherein the device emits delayed fluorescent light.

11. The organic light emitting device according to claim 9, wherein the device is an organic electroluminescent device.
